SEQUENCE LISTING (372 sequences)

SEQ ID NO: 1 - DNA, 21 bp, Artificial Sequence
  forward primer
atgtacgtcgccattcaagct

SEQ ID NO: 2 - DNA, 19 bp, Artificial Sequence
  reverse primer
acgccatcacctgaatcca

SEQ ID NO: 3 - DNA, 20 bp, Artificial Sequence
  probe
ctggccgcacgacaggcatc

SEQ ID NO: 4 - DNA, 20 bp, Artificial Sequence
  forward primer
cgcttctatggcgctgagat

SEQ ID NO: 5 - DNA, 20 bp, Artificial Sequence
  reverse primer
tcccggtacaccacgttctt

SEQ ID NO: 6 - DNA, 24 bp, Artificial Sequence
  probe
cagccctggactacctgcactcgg

SEQ ID NO: 7 - DNA, 22 bp, Artificial Sequence
  forward primer
ggctcttgtgcgtactgtcctt

SEQ ID NO: 8 - DNA, 23 bp, Artificial Sequence
  reverse primer
tcagatgacgaagagcacagatg

SEQ ID NO: 9 - DNA, 29 bp, Artificial Sequence
  probe
aggctcagtgatgtcttccctgtcaccag

SEQ ID NO: 10 - DNA, 19 bp, Artificial Sequence
  forward primer
gggtcaggtgcctcgagat

SEQ ID NO: 11 - DNA, 21 bp, Artificial Sequence
  reverse primer
ctgctcactcggctcaaactc

SEQ ID NO: 12 - DNA, 24 bp, Artificial Sequence
  probe
tgggcccagagcatgttccagatc

SEQ ID NO: 13 - DNA, 20 bp, Artificial Sequence
  forward primer
cctggagggtcctgtacaat

SEQ ID NO: 14 - DNA, 19 bp, Artificial Sequence
  reverse primer
ctaattgggctccatctcg

SEQ ID NO: 15 - DNA, 24 bp, Artificial Sequence
  probe
catcatgggactcctgcccttacc

SEQ ID NO: 16 - DNA, 25 bp, Artificial Sequence
  forward primer
cagatggacctagtacccactgaga

SEQ ID NO: 17 - DNA, 24 bp, Artificial Sequence
  reverse primer
cctatgatttaagggcatttttcc

SEQ ID NO: 18 - DNA, 22 bp, Artificial Sequence
  probe
ttccacgccgaaggacagcgat

SEQ ID NO: 19 - DNA, 24 bp, Artificial Sequence
  forward primer
cttttgtggaactctatgggaaca

SEQ ID NO: 20 - DNA, 19 bp, Artificial Sequence
  reverse primer
cagcggttgaagcgttcct

SEQ ID NO: 21 - DNA, 20 bp, Artificial Sequence
  probe
ttcggctctcggctgctgca

SEQ ID NO: 22 - DNA, 25 bp, Artificial Sequence
  forward primer
cagtttggcacaatcaataacttca

SEQ ID NO: 23 - DNA, 24 bp, Artificial Sequence
  reverse primer
gcagcattaatctcattccattcc

SEQ ID NO: 24 - DNA, 20 bp, Artificial Sequence
  probe
tcgcctgcccagtgttcccg

SEQ ID NO: 25 - DNA, 20 bp, Artificial Sequence
  forward primer
cctgcaaaagggaacaagag

SEQ ID NO: 26 - DNA, 20 bp, Artificial Sequence
  reverse primer
cgtggttgactctgatctcg

SEQ ID NO: 27 - DNA, 21 bp, Artificial Sequence
  probe
cttcgcctccagatggctccc

SEQ ID NO: 28 - DNA, 20 bp, Artificial Sequence
  forward primer
gtgcaggaaaggttcacaaa

SEQ ID NO: 29 - DNA, 20 bp, Artificial Sequence
  reverse primer
gcacacacgatggagtaagg

SEQ ID NO: 30 - DNA, 24 bp, Artificial Sequence
  probe
agtgtctgcgtccaatacacgcgt

SEQ ID NO: 31 - DNA, 20 bp, Artificial Sequence
  forward primer
tcagctgtgagctgcggata

SEQ ID NO: 32 - DNA, 24 bp, Artificial Sequence
  reverse primer
acggtcctaggtttgaggttaaga

SEQ ID NO: 33 - DNA, 19 bp, Artificial Sequence
  probe
caggtcccattgccgggcg

SEQ ID NO: 34 - DNA, 20 bp, Artificial Sequence
  forward primer
atcctagccctggtttttgg

SEQ ID NO: 35 - DNA, 20 bp, Artificial Sequence
  reverse primer
ctgccttctcatctgcacaa

SEQ ID NO: 36 - DNA, 20 bp, Artificial Sequence
  probe
tttgctgtcaccagcgtcgc

SEQ ID NO: 37 - DNA, 20 bp, Artificial Sequence
  forward primer
ttcaggttgttgcaggagac

SEQ ID NO: 38 - DNA, 20 bp, Artificial Sequence
  reverse primer
catcttcttgggcacacaat

SEQ ID NO: 39 - DNA, 27 bp, Artificial Sequence
  probe
tgtctccattattgatcggttcatgca

SEQ ID NO: 40 - DNA, 21 bp, Artificial Sequence
  forward primer
gcatgttcgtggcctctaaga

SEQ ID NO: 41 - DNA, 22 bp, Artificial Sequence
  reverse primer
cggtgtagatgcacagcttctc

SEQ ID NO: 42 - DNA, 23 bp, Artificial Sequence
  probe
aaggagaccatccccctgacggc

SEQ ID NO: 43 - DNA, 25 bp, Artificial Sequence
  forward primer
tgtatttcaagacctctgtgcactt

SEQ ID NO: 44 - DNA, 23 bp, Artificial Sequence
  reverse primer
ttagcctgaggaattgctgtgtt

SEQ ID NO: 45 - DNA, 25 bp, Artificial Sequence
  probe
tttatgaacctgccctgctcccaca

SEQ ID NO: 46 - DNA, 20 bp, Artificial Sequence
  forward primer
agatgaagtggaaggcgctt

SEQ ID NO: 47 - DNA, 21 bp, Artificial Sequence
  reverse primer
tgcctctgtaatcggcaactg

SEQ ID NO: 48 - DNA, 18 bp, Artificial Sequence
  probe
caccgcggccatcctgca

SEQ ID NO: 49 - DNA, 20 bp, Artificial Sequence
  forward primer
gggcgtggaacagtttatct

SEQ ID NO: 50 - DNA, 19 bp, Artificial Sequence
  reverse primer
cacggtgaaggtttcgagt

SEQ ID NO: 51 - DNA, 24 bp, Artificial Sequence
  probe
agacatctgccccaagaaggacgt

SEQ ID NO: 52 - DNA, 21 bp, Artificial Sequence
  forward primer
tggattggagttctgggaatg

SEQ ID NO: 53 - DNA, 22 bp, Artificial Sequence
  reverse primer
gcttgcactccacaggtacaca

SEQ ID NO: 54 - DNA, 23 bp, Artificial Sequence
  probe
actggccgtggcactggacaaca

SEQ ID NO: 55 - DNA, 21 bp, Artificial Sequence
  forward primer
tgagtgtcccccggtatcttc

SEQ ID NO: 56 - DNA, 21 bp, Artificial Sequence
  reverse primer
cagccgctttcagattttcat

SEQ ID NO: 57 - DNA, 27 bp, Artificial Sequence
  probe
tgccaatcccgatgaaattggaaattt

SEQ ID NO: 58 - DNA, 21 bp, Artificial Sequence
  forward primer
tgacaatcagcacacctgcat

SEQ ID NO: 59 - DNA, 23 bp, Artificial Sequence
  reverse primer
tgtgactacagccgtgatcctta

SEQ ID NO: 60 - DNA, 20 bp, Artificial Sequence
  probe
caggccctcttccgagcggt

SEQ ID NO: 61 - DNA, 21 bp, Artificial Sequence
  forward primer
atgtggaacccccacctactt

SEQ ID NO: 62 - DNA, 22 bp, Artificial Sequence
  reverse primer
cagtccacagcacggttatacc

SEQ ID NO: 63 - DNA, 29 bp, Artificial Sequence
  probe
agtcccaacagaaacaagaacttcaggcg

SEQ ID NO: 64 - DNA, 24 bp, Artificial Sequence
  forward primer
ggatatttccgtggctcttattca

SEQ ID NO: 65 - DNA, 25 bp, Artificial Sequence
  reverse primer
cttctcatcaaggcagaaaaatctt

SEQ ID NO: 66 - DNA, 30 bp, Artificial Sequence
  probe
tctccatcaaatcctgtaaactccagagca

SEQ ID NO: 67 - DNA, 22 bp, Artificial Sequence
  forward primer
gacatttccagtcctgcagtca

SEQ ID NO: 68 - DNA, 20 bp, Artificial Sequence
  reverse primer
ctccgatcgcacacatttgt

SEQ ID NO: 69 - DNA, 23 bp, Artificial Sequence
  probe
tgcctctctgccccaccctttgt

SEQ ID NO: 70 - DNA, 18 bp, Artificial Sequence
  forward primer
tccaccctcctggctttg

SEQ ID NO: 71 - DNA, 21 bp, Artificial Sequence
  reverse primer
tcactcccacgttcaccttgt

SEQ ID NO: 72 - DNA, 23 bp, Artificial Sequence
  probe
tcctttcgtcttcgccatgctgg

SEQ ID NO: 73 - DNA, 18 bp, Artificial Sequence
  forward primer
gtggccatccagctgacc

SEQ ID NO: 74 - DNA, 23 bp, Artificial Sequence
  reverse primer
cagtggtaggtgatgttctggga

SEQ ID NO: 75 - DNA, 21 bp, Artificial Sequence
  probe
tcctgcgcctgatgtccaccg

SEQ ID NO: 76 - DNA, 24 bp, Artificial Sequence
  forward primer
cagccaagaactggtataggagct

SEQ ID NO: 77 - DNA, 19 bp, Artificial Sequence
  reverse primer
aaactggctgccagcattg

SEQ ID NO: 78 - DNA, 30 bp, Artificial Sequence
  probe
tctcctagccagacgtgtttcttgtccttg

SEQ ID NO: 79 - DNA, 23 bp, Artificial Sequence
  forward primer
tctgcagagttggaagcactcta

SEQ ID NO: 80 - DNA, 21 bp, Artificial Sequence
  reverse primer
gccgaggcttttctaccagaa

SEQ ID NO: 81 - DNA, 28 bp, Artificial Sequence
  probe
caggatacagctccacagcatcgatgtc

SEQ ID NO: 82 - DNA, 21 bp, Artificial Sequence
  forward primer
tgtctcactgagcgagcagaa

SEQ ID NO: 83 - DNA, 19 bp, Artificial Sequence
  reverse primer
accattgcagccctgattg

SEQ ID NO: 84 - DNA, 24 bp, Artificial Sequence
  probe
cttgaggacgcgaacagtccacca

SEQ ID NO: 85 - DNA, 20 bp, Artificial Sequence
  forward primer
ccgtgttcaagaggaagctc

SEQ ID NO: 86 - DNA, 20 bp, Artificial Sequence
  reverse primer
agtgggatcacagggtgaag

SEQ ID NO: 87 - DNA, 24 bp, Artificial Sequence
  probe
ttttctcaactcctccacaaggca

SEQ ID NO: 88 - DNA, 22 bp, Artificial Sequence
  forward primer
gggagaacgggatcaataggat

SEQ ID NO: 89 - DNA, 21 bp, Artificial Sequence
  reverse primer
gcatcagccagtcctcaaact

SEQ ID NO: 90 - DNA, 24 bp, Artificial Sequence
  probe
ctcattgggcaccagcaggtttcc

SEQ ID NO: 91 - DNA, 19 bp, Artificial Sequence
  forward primer
cacaatggcggctctgaag

SEQ ID NO: 92 - DNA, 26 bp, Artificial Sequence
  reverse primer
acacaaacactgtctgtacctgaaga

SEQ ID NO: 93 - DNA, 23 bp, Artificial Sequence
  probe
aagttacgctgcgcgacagccaa

SEQ ID NO: 94 - DNA, 20 bp, Artificial Sequence
  forward primer
cagtgcttccatggacaagt

SEQ ID NO: 95 - DNA, 20 bp, Artificial Sequence
  reverse primer
tggacagggatgattgatgt

SEQ ID NO: 96 - DNA, 24 bp, Artificial Sequence
  probe
atctccatcagcatgggccagttt

SEQ ID NO: 97 - DNA, 24 bp, Artificial Sequence
  forward primer
ctctgagacagtgcttcgatgact

SEQ ID NO: 98 - DNA, 19 bp, Artificial Sequence
  reverse primer
ccatgaggcccaacttcct

SEQ ID NO: 99 - DNA, 23 bp, Artificial Sequence
  probe
cagacttggtgccctttgactcc

SEQ ID NO: 100 - DNA, 20 bp, Artificial Sequence
  forward primer
tgtcgatggacttccagaac

SEQ ID NO: 101 - DNA, 19 bp, Artificial Sequence
  reverse primer
attgggacagcttggatca

SEQ ID NO: 102 - DNA, 18 bp, Artificial Sequence
  probe
cacctgggcagctgccaa

SEQ ID NO: 103 - DNA, 18 bp, Artificial Sequence
  forward primer
aagccgcggttgaatgtg

SEQ ID NO: 104 - DNA, 20 bp, Artificial Sequence
  reverse primer
tgacgccagcttcaatgatg

SEQ ID NO: 105 - DNA, 25 bp, Artificial Sequence
  probe
tgaccctctccctctctggatggca

SEQ ID NO: 106 - DNA, 20 bp, Artificial Sequence
  forward primer
accgtaggctctgctctgaa

SEQ ID NO: 107 - DNA, 20 bp, Artificial Sequence
  reverse primer
tggtccaggtggaaaacttc

SEQ ID NO: 108 - DNA, 20 bp, Artificial Sequence
  probe
aggcagccagacccacagga

SEQ ID NO: 109 - DNA, 23 bp, Artificial Sequence
  forward primer
cggttatgtcatgccagatacac

SEQ ID NO: 110 - DNA, 24 bp, Artificial Sequence
  reverse primer
gaactgagacccactgaagaaagg

SEQ ID NO: 111 - DNA, 25 bp, Artificial Sequence
  probe
cctcaaaggtactccctcctcccgg

SEQ ID NO: 112 - DNA, 19 bp, Artificial Sequence
  forward primer
cgtggtgcccctctatgac

SEQ ID NO: 113 - DNA, 19 bp, Artificial Sequence
  reverse primer
ggctagtgggcgcatgtag

SEQ ID NO: 114 - DNA, 19 bp, Artificial Sequence
  probe
ctggagatgctggacgccc

SEQ ID NO: 115 - DNA, 20 bp, Artificial Sequence
  forward primer
cacgggacattcaccacatc

SEQ ID NO: 116 - DNA, 19 bp, Artificial Sequence
  reverse primer
gggtgccatccacttcaca

SEQ ID NO: 117 - DNA, 27 bp, Artificial Sequence
  probe
ataaaaagacaaccaacggccgactgc

SEQ ID NO: 118 - DNA, 21 bp, Artificial Sequence
  forward primer
gcgtatgatttcccgaatgag

SEQ ID NO: 119 - DNA, 21 bp, Artificial Sequence
  reverse primer
cagtgacctcgtacccattgc

SEQ ID NO: 120 - DNA, 25 bp, Artificial Sequence
  probe
atgttgatatgcccaaacttcatga

SEQ ID NO: 121 - DNA, 19 bp, Artificial Sequence
  forward primer
tcagcagcaagggcatcat

SEQ ID NO: 122 - DNA, 23 bp, Artificial Sequence
  reverse primer
ggtggttttcttgagcgtgtact

SEQ ID NO: 123 - DNA, 19 bp, Artificial Sequence
  probe
cgcccgcaggcctcatcct

SEQ ID NO: 124 - DNA, 23 bp, Artificial Sequence
  forward primer
caaaggagctcactgtggtgtct

SEQ ID NO: 125 - DNA, 26 bp, Artificial Sequence
  reverse primer
gagtcagaatggcttattcacagatg

SEQ ID NO: 126 - DNA, 24 bp, Artificial Sequence
  probe
tgttccaaccactgaatctggacc

SEQ ID NO: 127 - DNA, 22 bp, Artificial Sequence
  forward primer
cttctgctaagcggtacatcga

SEQ ID NO: 128 - DNA, 20 bp, Artificial Sequence
  reverse primer
ggctcaaagccttgcttcac

SEQ ID NO: 129 - DNA, 21 bp, Artificial Sequence
  probe
acccagccaatcgggatcggc

SEQ ID NO: 130 - DNA, 20 bp, Artificial Sequence
  forward primer
gagaccctgctgtcccagaa

SEQ ID NO: 131 - DNA, 23 bp, Artificial Sequence
  reverse primer
ggttgtagtcagcgaaggagatc

SEQ ID NO: 132 - DNA, 26 bp, Artificial Sequence
  probe
tcccacaatgaaggtcttgcctccct

SEQ ID NO: 133 - DNA, 20 bp, Artificial Sequence
  forward primer
cggtgtgagaagtgcagcaa

SEQ ID NO: 134 - DNA, 19 bp, Artificial Sequence
  reverse primer
cctctcgcaagtgctccat

SEQ ID NO: 135 - DNA, 24 bp, Artificial Sequence
  probe
ccagaccatagcacactcgggcac

SEQ ID NO: 136 - DNA, 24 bp, Artificial Sequence
  forward primer
tgaacataaagtctgcaacatgga

SEQ ID NO: 137 - DNA, 28 bp, Artificial Sequence
  reverse primer
tgaggttggttactgttggtatcatata

SEQ ID NO: 138 - DNA, 24 bp, Artificial Sequence
  probe
ttgcactgcacaggccacattcac

SEQ ID NO: 139 - DNA, 21 bp, Artificial Sequence
  forward primer
tccatgatggttctgcaggtt

SEQ ID NO: 140 - DNA, 21 bp, Artificial Sequence
  reverse primer
tgagcagcaccatcagtaacg

SEQ ID NO: 141 - DNA, 21 bp, Artificial Sequence
  probe
ccccggacagtggctctgacg

SEQ ID NO: 142 - DNA, 24 bp, Artificial Sequence
  forward primer
tccaggatgttaggaactgtgaag

SEQ ID NO: 143 - DNA, 22 bp, Artificial Sequence
  reverse primer
gcgtgtctgcgtagtagctgtt

SEQ ID NO: 144 - DNA, 24 bp, Artificial Sequence
  probe
agtcgctggtttcatgcccttcca

SEQ ID NO: 145 - DNA, 19 bp, Artificial Sequence
  forward primer
agaaccgcaaggtgagcaa

SEQ ID NO: 146 - DNA, 21 bp, Artificial Sequence
  reverse primer
tccaactgaaggtccctgatg

SEQ ID NO: 147 - DNA, 26 bp, Artificial Sequence
  probe
tggagattctccagcacgtcatcgac

SEQ ID NO: 148 - DNA, 23 bp, Artificial Sequence
  forward primer
aacgactgctactccaagctcaa

SEQ ID NO: 149 - DNA, 22 bp, Artificial Sequence
  reverse primer
ggatttccatcttgctcacctt

SEQ ID NO: 150 - DNA, 22 bp, Artificial Sequence
  probe
tgcccagcatcccccagaacaa

SEQ ID NO: 151 - DNA, 21 bp, Artificial Sequence
  forward primer
gcatggtagccgaagatttca

SEQ ID NO: 152 - DNA, 30 bp, Artificial Sequence
  reverse primer
tttccggtaatagtctgtctcatagatatc

SEQ ID NO: 153 - DNA, 28 bp, Artificial Sequence
  probe
cgcgtcataccaaaatctccgattttga

SEQ ID NO: 154 - DNA, 19 bp, Artificial Sequence
  forward primer
gtggacagcaccatgaaca

SEQ ID NO: 155 - DNA, 20 bp, Artificial Sequence
  reverse primer
ccttcatacccgacttgagg

SEQ ID NO: 156 - DNA, 20 bp, Artificial Sequence
  probe
cttccggccagcactgcctc

SEQ ID NO: 157 - DNA, 20 bp, Artificial Sequence
  forward primer
ccacagctcaccttctgtca

SEQ ID NO: 158 - DNA, 20 bp, Artificial Sequence
  reverse primer
cctcagtgccagtctcttcc

SEQ ID NO: 159 - DNA, 20 bp, Artificial Sequence
  probe
tccatcccagctccagccag

SEQ ID NO: 160 - DNA, 19 bp, Artificial Sequence
  forward primer
cggactttgggtgcgactt

SEQ ID NO: 161 - DNA, 24 bp, Artificial Sequence
  reverse primer
ttacaactcttccactgggacgat

SEQ ID NO: 162 - DNA, 23 bp, Artificial Sequence
  probe
ccacttgtcgaaccaccgctcgt

SEQ ID NO: 163 - DNA, 24 bp, Artificial Sequence
  forward primer
gcctagcagttctaccatgatcag

SEQ ID NO: 164 - DNA, 22 bp, Artificial Sequence
  reverse primer
ggtgatcggtccagatgtttct

SEQ ID NO: 165 - DNA, 22 bp, Artificial Sequence
  probe
agagctccacccgctcgaagca

SEQ ID NO: 166 - DNA, 18 bp, Artificial Sequence
  forward primer
gcccagaggctccatcgt

SEQ ID NO: 167 - DNA, 23 bp, Artificial Sequence
  reverse primer
cagaggtttgaacagtgcagaca

SEQ ID NO: 168 - DNA, 23 bp, Artificial Sequence
  probe
cctcttcctccccagtcggctga

SEQ ID NO: 169 - DNA, 20 bp, Artificial Sequence
  forward primer
ggcctgctgagatcaaagac

SEQ ID NO: 170 - DNA, 20 bp, Artificial Sequence
  reverse primer
gtccactgtggctgtgagaa

SEQ ID NO: 171 - DNA, 26 bp, Artificial Sequence
  probe
tgttcctcaggtcctcaatggtcttg

SEQ ID NO: 172 - DNA, 21 bp, Artificial Sequence
  forward primer
cgaggattggttcttcagcaa

SEQ ID NO: 173 - DNA, 22 bp, Artificial Sequence
  reverse primer
actctgcaccagctcactgttg

SEQ ID NO: 174 - DNA, 24 bp, Artificial Sequence
  probe
cacctcgcggttcagttcctctgt

SEQ ID NO: 175 - DNA, 20 bp, Artificial Sequence
  forward primer
agagatcgaggctctcaagg

SEQ ID NO: 176 - DNA, 20 bp, Artificial Sequence
  reverse primer
ggccttttacttcctcttcg

SEQ ID NO: 177 - DNA, 27 bp, Artificial Sequence
  probe
tggttcttcttcatgaagagcagctcc

SEQ ID NO: 178 - DNA, 21 bp, Artificial Sequence
  forward primer
tgagcggcagaatcaggagta

SEQ ID NO: 179 - DNA, 19 bp, Artificial Sequence
  reverse primer
tgcggtaggtggcaatctc

SEQ ID NO: 180 - DNA, 24 bp, Artificial Sequence
  probe
ctcatggacatcaagtcgcggctg

SEQ ID NO: 181 - DNA, 20 bp, Artificial Sequence
  forward primer
tcagtggagaaggagttgga

SEQ ID NO: 182 - DNA, 20 bp, Artificial Sequence
  reverse primer
tgccatatccagaggaaaca

SEQ ID NO: 183 - DNA, 28 bp, Artificial Sequence
  probe
ccagtcaacatctctgttgtcacaagca

SEQ ID NO: 184 - DNA, 21 bp, Artificial Sequence
  forward primer
gacttttgcccgctacctttc

SEQ ID NO: 185 - DNA, 26 bp, Artificial Sequence
  reverse primer
gccactaactgcttcagtatgaagag

SEQ ID NO: 186 - DNA, 24 bp, Artificial Sequence
  probe
acagctcattgttgtcacgccgga

SEQ ID NO: 187 - DNA, 20 bp, Artificial Sequence
  forward primer
ggagaacaatccccttgaga

SEQ ID NO: 188 - DNA, 20 bp, Artificial Sequence
  reverse primer
atctcctggatggtgatggt

SEQ ID NO: 189 - DNA, 27 bp, Artificial Sequence
  probe
tggcctttctgtctacaaggatcacca

SEQ ID NO: 190 - DNA, 19 bp, Artificial Sequence
  forward primer
ctacagggacgccatcgaa

SEQ ID NO: 191 - DNA, 23 bp, Artificial Sequence
  reverse primer
atccaaccaatcacctgaatgtt

SEQ ID NO: 192 - DNA, 23 bp, Artificial Sequence
  probe
cttacaccagcatcaagatccgg

SEQ ID NO: 193 - DNA, 20 bp, Artificial Sequence
  forward primer
ccatgatggagaggcagaca

SEQ ID NO: 194 - DNA, 21 bp, Artificial Sequence
  reverse primer
ggagtccgtccttaccgtcaa

SEQ ID NO: 195 - DNA, 24 bp, Artificial Sequence
  probe
ctgggagcatggcgatggataccc

SEQ ID NO: 196 - DNA, 20 bp, Artificial Sequence
  forward primer
gagaaccaatctcaccgaca

SEQ ID NO: 197 - DNA, 20 bp, Artificial Sequence
  reverse primer
cacccgagtgtaaccatagc

SEQ ID NO: 198 - DNA, 24 bp, Artificial Sequence
  probe
acaggtattcctctgccagctgcc

SEQ ID NO: 199 - DNA, 22 bp, Artificial Sequence
  forward primer
acgagaacgagggcatctatgt

SEQ ID NO: 200 - DNA, 22 bp, Artificial Sequence
  reverse primer
gcatgtaggtgcttccaatcac

SEQ ID NO: 201 - DNA, 25 bp, Artificial Sequence
  probe
cgcacctttccggtcttgacatcct

SEQ ID NO: 202 - DNA, 27 bp, Artificial Sequence
  forward primer
cggtacttctcagggctaatatatacg

SEQ ID NO: 203 - DNA, 21 bp, Artificial Sequence
  reverse primer
ccgagtagatgggcaggtgtt

SEQ ID NO: 204 - DNA, 26 bp, Artificial Sequence
  probe
ctcttctgcgtggtggtcaaccccta

SEQ ID NO: 205 - DNA, 18 bp, Artificial Sequence
  forward primer
gtgaggcagcgcgactct

SEQ ID NO: 206 - DNA, 23 bp, Artificial Sequence
  reverse primer
tgccaatggtgtacaacacttca

SEQ ID NO: 207 - DNA, 21 bp, Artificial Sequence
  probe
tgccttcccgggctgaggact

SEQ ID NO: 208 - DNA, 19 bp, Artificial Sequence
  forward primer
ctgccgggatggcttctat

SEQ ID NO: 209 - DNA, 22 bp, Artificial Sequence
  reverse primer
ccaggttctggaaactgtggat

SEQ ID NO: 210 - DNA, 21 bp, Artificial Sequence
  probe
ctgagctctgcccggaccgct

SEQ ID NO: 211 - DNA, 20 bp, Artificial Sequence
  forward primer
ggctgtggctgaggctgtag

SEQ ID NO: 212 - DNA, 21 bp, Artificial Sequence
  reverse primer
ggagcattcgaggtcaaatca

SEQ ID NO: 213 - DNA, 28 bp, Artificial Sequence
  probe
ttcccagagtgtctcacctccagcagag

SEQ ID NO: 214 - DNA, 20 bp, Artificial Sequence
  forward primer
ccagctctccttccagctac

SEQ ID NO: 215 - DNA, 20 bp, Artificial Sequence
  reverse primer
gggtggctctcacttagctc

SEQ ID NO: 216 - DNA, 24 bp, Artificial Sequence
  probe
atcaatgtccctgtccgagtgctg

SEQ ID NO: 217 - DNA, 20 bp, Artificial Sequence
  forward primer
cccatggatgctcctctgaa

SEQ ID NO: 218 - DNA, 20 bp, Artificial Sequence
  reverse primer
ccggtggctaccagacattg

SEQ ID NO: 219 - DNA, 22 bp, Artificial Sequence
  probe
cattgactgccgaggccccatg

SEQ ID NO: 220 - DNA, 20 bp, Artificial Sequence
  forward primer
gcatcaggctgtcattatgg

SEQ ID NO: 221 - DNA, 20 bp, Artificial Sequence
  reverse primer
agtagttgtgctgcccttcc

SEQ ID NO: 222 - DNA, 28 bp, Artificial Sequence
  probe
tgtccttacctgtgggagctgtaaggtc

SEQ ID NO: 223 - DNA, 19 bp, Artificial Sequence
  forward primer
gccctcccagtgtgcaaat

SEQ ID NO: 224 - DNA, 25 bp, Artificial Sequence
  reverse primer
cgtcgatggtattaggatagaagca

SEQ ID NO: 225 - DNA, 23 bp, Artificial Sequence
  probe
tgctgtttcgacgacaccgttcg

SEQ ID NO: 226 - DNA, 18 bp, Artificial Sequence
  forward primer
gggacactgcgggacaag

SEQ ID NO: 227 - DNA, 18 bp, Artificial Sequence
  reverse primer
gcccatggcgtctctgaa

SEQ ID NO: 228 - DNA, 25 bp, Artificial Sequence
  probe
cggttccggagtctcaccactgcat

SEQ ID NO: 229 - DNA, 23 bp, Artificial Sequence
  forward primer
agctagcctcagtgacacacatg

SEQ ID NO: 230 - DNA, 18 bp, Artificial Sequence
  reverse primer
ccggatctgacggctgtt

SEQ ID NO: 231 - DNA, 24 bp, Artificial Sequence
  probe
acacaacgtcggcagtgcaacctg

SEQ ID NO: 232 - DNA, 20 bp, Artificial Sequence
  forward primer
cgaagcccttacaagtttcc

SEQ ID NO: 233 - DNA, 20 bp, Artificial Sequence
  reverse primer
ggactcttcaggggtgaaat

SEQ ID NO: 234 - DNA, 24 bp, Artificial Sequence
  probe
cccttacggattcctggagggaac

SEQ ID NO: 235 - DNA, 20 bp, Artificial Sequence
  forward primer
ccagacgagcgattagaagc

SEQ ID NO: 236 - DNA, 20 bp, Artificial Sequence
  reverse primer
tcctcctcttcctcctcctc

SEQ ID NO: 237 - DNA, 23 bp, Artificial Sequence
  probe
tgtgaggtgaatgatttggggga

SEQ ID NO: 238 - DNA, 25 bp, Artificial Sequence
  forward primer
gctcattatgaaaaacatcccaaac

SEQ ID NO: 239 - DNA, 26 bp, Artificial Sequence
  reverse primer
aagaaacagaagttgtctggctttct

SEQ ID NO: 240 - DNA, 24 bp, Artificial Sequence
  probe
cacaccaaccaataatttcgcatt

SEQ ID NO: 241 - DNA, 20 bp, Artificial Sequence
  forward primer
catcttccaggaggaccact

SEQ ID NO: 242 - DNA, 20 bp, Artificial Sequence
  reverse primer
tccgaccttcaatcatttca

SEQ ID NO: 243 - DNA, 24 bp, Artificial Sequence
  probe
ctctgtggcaccctggactacctg

SEQ ID NO: 244 - DNA, 20 bp, Artificial Sequence
  forward primer
cctggaggctgcaacatacc

SEQ ID NO: 245 - DNA, 23 bp, Artificial Sequence
  reverse primer
tacaatggctttggaggatagca

SEQ ID NO: 246 - DNA, 25 bp, Artificial Sequence
  probe
atcctcctgaagcccttttcgcagc

SEQ ID NO: 247 - DNA, 20 bp, Artificial Sequence
  forward primer
tgttttgattcccgggctta

SEQ ID NO: 248 - DNA, 24 bp, Artificial Sequence
  reverse primer
caaagctgtcagctctagcaaaag

SEQ ID NO: 249 - DNA, 28 bp, Artificial Sequence
  probe
tgccttcttcctccctcacttctcacct

SEQ ID NO: 250 - DNA, 22 bp, Artificial Sequence
  forward primer
ggatcgagctcttccagatcct

SEQ ID NO: 251 - DNA, 20 bp, Artificial Sequence
  reverse primer
gccaccgatatagcgctgtt

SEQ ID NO: 252 - DNA, 21 bp, Artificial Sequence
  probe
cggccagatgagcacattgcc

SEQ ID NO: 253 - DNA, 22 bp, Artificial Sequence
  forward primer
tcaccctctgtgacttcatcgt

SEQ ID NO: 254 - DNA, 22 bp, Artificial Sequence
  reverse primer
tgtggttcaggctcttcttctg

SEQ ID NO: 255 - DNA, 22 bp, Artificial Sequence
  probe
ccctgggacaccctgagcacca

SEQ ID NO: 256 - DNA, 20 bp, Artificial Sequence
  forward primer
ctacctgccttgctttgtga

SEQ ID NO: 257 - DNA, 20 bp, Artificial Sequence
  reverse primer
accgaaattggagagcatgt

SEQ ID NO: 258 - DNA, 24 bp, Artificial Sequence
  probe
ccaagaacgagtgtctctggaccg

SEQ ID NO: 259 - DNA, 20 bp, Artificial Sequence
  forward primer
aatccaagggggagagtgat

SEQ ID NO: 260 - DNA, 20 bp, Artificial Sequence
  reverse primer
gtacagattttgcccgagga

SEQ ID NO: 261 - DNA, 26 bp, Artificial Sequence
  probe
catatggactttgactcagctgtggc

SEQ ID NO: 262 - DNA, 20 bp, Artificial Sequence
  forward primer
tgctgttgctgagtctgttg

SEQ ID NO: 263 - DNA, 20 bp, Artificial Sequence
  reverse primer
cttgcctggcttcacagata

SEQ ID NO: 264 - DNA, 24 bp, Artificial Sequence
  probe
ccagtccccagaagaccatgtctg

SEQ ID NO: 265 - DNA, 20 bp, Artificial Sequence
  forward primer
ctgctgtcttgggtgcattg

SEQ ID NO: 266 - DNA, 18 bp, Artificial Sequence
  reverse primer
gcagcctgggaccacttg

SEQ ID NO: 267 - DNA, 25 bp, Artificial Sequence
  probe
ttgccttgctgctctacctccacca

SEQ ID NO: 268 - DNA, 19 bp, Artificial Sequence
  forward primer
tgacgatggcctggagtgt

SEQ ID NO: 269 - DNA, 22 bp, Artificial Sequence
  reverse primer
ggtaccggatcatgaggatctg

SEQ ID NO: 270 - DNA, 21 bp, Artificial Sequence
  probe
ctgggcagcaccaagtccgga

SEQ ID NO: 271 - DNA, 25 bp, Artificial Sequence
  forward primer
cctcagcaagacgttatttgaaatt

SEQ ID NO: 272 - DNA, 24 bp, Artificial Sequence
  reverse primer
aagtgtgattggcaaaactgattg

SEQ ID NO: 273 - DNA, 26 bp, Artificial Sequence
  probe
cctctctctcaaggccccaaaccagt

SEQ ID NO: 274 - DNA, 21 bp, Artificial Sequence
  forward primer
tgcccttaaaggaaccaatga

SEQ ID NO: 275 - DNA, 22 bp, Artificial Sequence
  reverse primer
gcttcaacggcaaagttctctt

SEQ ID NO: 276 - DNA, 22 bp, Artificial Sequence
  probe
atttcacgcatctggcgttcca

SEQ ID NO: 277 - DNA, 20 bp, Artificial Sequence
  forward primer
acccagtagcaaggagaagc

SEQ ID NO: 278 - DNA, 20 bp, Artificial Sequence
  reverse primer
cagctggtggtaggttctga

SEQ ID NO: 279 - DNA, 21 bp, Artificial Sequence
  probe
cactcactgctccgagtgcgg

SEQ ID NO: 280 - DNA, 83 bp, Artificial Sequence
  amplicon
atgtacgtcgccattcaagctgtgctctccctctatgcctctggccgcacgacaggcatcgtcctggattcaggtgatggcgt

SEQ ID NO: 281 - DNA, 71 bp, Artificial Sequence
  amplicon
cgcttctatggcgctgagattgtgtcagccctggactacctgcactcggagaagaacgtggtgtaccggga

SEQ ID NO: 282 - DNA, 80 bp, Artificial Sequence
  amplicon
ggctcttgtgcgtactgtccttcgggctggtgacagggaagacatcactgagcctgccatctgtgctcttcgtcatctga

SEQ ID NO: 283 - DNA, 73 bp, Artificial Sequence
  amplicon
gggtcaggtgcctcgagatcgggcttgggcccagagcatgttccagatcccagagtttgagccgagtgagcag

SEQ ID NO: 284 - DNA, 83 bp, Artificial Sequence
  amplicon
cctggagggtcctgtacaatctcatcatgggactcctgcccttacccaggggccacagagcccccgagatggagcccaattag

SEQ ID NO: 285 - DNA, 73 bp, Artificial Sequence
  amplicon
cagatggacctagtacccactgagatttccacgccgaaggacagcgatgggaaaaatgcccttaaatcatagg

SEQ ID NO: 286 - DNA, 70 bp, Artificial Sequence
  amplicon
cttttgtggaactctatgggaacaatgcagcagccgagagccgaaagggccaggaacgcttcaaccgctg

SEQ ID NO: 287 - DNA, 77 bp, Artificial Sequence
  amplicon
cagtttggcacaatcaataacttcaggctgggtcgcctgcccagtgttcccgtggaatggaatgagattaatgctgc

SEQ ID NO: 288 - DNA, 76 bp, Artificial Sequence
  amplicon
cctgcaaaagggaacaagagcccttcgcctccagatggctcccctgccgccacccccgagatcagagtcaaccacg

SEQ ID NO: 289 - DNA, 79 bp, Artificial Sequence
  amplicon
gtgcaggaaaggttcacaaatgtggagtgtctgcgtccaatacacgcgtgtgctcctctccttactccatcgtgtgtgc

SEQ ID NO: 290 - DNA, 65 bp, Artificial Sequence
  amplicon
tcagctgtgagctgcggataccgcccggcaatgggacctgctcttaacctcaaacctaggaccgt

SEQ ID NO: 291 - DNA, 72 bp, Artificial Sequence
  amplicon
atcctagccctggtttttggcctcctttttgctgtcaccagcgtcgcgttccttgtgcagatgagaaggcag

SEQ ID NO: 292 - DNA, 84 bp, Artificial Sequence
  amplicon
ttcaggttgttgcaggagaccatgtacatgactgtctccattattgatcggttcatgcagaataattgtgtgcccaagaagatg

SEQ ID NO: 293 - DNA, 69 bp, Artificial Sequence
  amplicon
gcatgttcgtggcctctaagatgaaggagaccatccccctgacggccgagaagctgtgcatctacaccg

SEQ ID NO: 294 - DNA, 75 bp, Artificial Sequence
  amplicon
tgtatttcaagacctctgtgcacttatttatgaacctgccctgctcccacagaacacagcaattcctcaggctaa

SEQ ID NO: 295 - DNA, 65 bp, Artificial Sequence
  amplicon
agatgaagtggaaggcgcttttcaccgcggccatcctgcaggcacagttgccgattacagaggca

SEQ ID NO: 296 - DNA, 64 bp, Artificial Sequence
  amplicon
gggcgtggaacagtttatctcagacatctgccccaagaaggacgtactcgaaaccttcaccgtg

SEQ ID NO: 297 - DNA, 68 bp, Artificial Sequence
  amplicon
tggattggagttctgggaatgtactggccgtggcactggacaacagtgtgtacctgtggagtgcaagc

SEQ ID NO: 298 - DNA, 81 bp, Artificial Sequence
  amplicon
tgagtgtcccccggtatcttccccgccctgccaatcccgatgaaattggaaattttattgatgaaaatctgaaagcggctg

SEQ ID NO: 299 - DNA, 77 bp, Artificial Sequence
  amplicon
tgacaatcagcacacctgcattcaccgctcggaagagggcctgagctgcatgaataaggatcacggctgtagtcaca

SEQ ID NO: 300 - DNA, 78 bp, Artificial Sequence
  amplicon
atgtggaacccccacctacttggcgcctgaagttcttgtttctgttgggactgctgggtataaccgtgctgtggactg

SEQ ID NO: 301 - DNA, 86 bp, Artificial Sequence
  amplicon
ggatatttccgtggctcttattcaaactctccatcaaatcctgtaaactccagagcaaatcaagatttttctgccttgatgagaag

SEQ ID NO: 302 - DNA, 86 bp, Artificial Sequence
  amplicon
gacatttccagtcctgcagtcaatgcctctctgccccaccctttgttcagtgtggctggtgccacgacaaatgtgtgcgatcggag

SEQ ID NO: 303 - DNA, 64 bp, Artificial Sequence
  amplicon
tccaccctcctggctttggccagcatggcgaagacgaaaggaaacaaggtgaacgtgggagtga

SEQ ID NO: 304 - DNA, 68 bp, Artificial Sequence
  amplicon
gtggccatccagctgaccttcctgcgcctgatgtccaccgaggcctcccagaacatcacctaccactg

SEQ ID NO: 305 - DNA, 80 bp, Artificial Sequence
  amplicon
cagccaagaactggtataggagctccaaggacaagaaacacgtctggctaggagaaactatcaatgctggcagccagttt

SEQ ID NO: 306 - DNA, 79 bp, Artificial Sequence
  amplicon
tctgcagagttggaagcactctatggtgacatcgatgctgtggagctgtatcctgcccttctggtagaaaagcctcggc

SEQ ID NO: 307 - DNA, 67 bp, Artificial Sequence
  amplicon
tgtctcactgagcgagcagaatctggtggactgttcgcgtcctcaaggcaatcagggctgcaatggt

SEQ ID NO: 308 - DNA, 73 bp, Artificial Sequence
  amplicon
ccgtgttcaagaggaagctcactgccttgtggaggagttgagaaaaaccaaggcttcaccctgtgatcccact

SEQ ID NO: 309 - DNA, 78 bp, Artificial Sequence
  amplicon
gggagaacgggatcaataggatcggaaacctgctggtgcccaatgagaattactgcaagtttgaggactggctgatgc

SEQ ID NO: 310 - DNA, 73 bp, Artificial Sequence
  amplicon
cacaatggcggctctgaagagttggctgtcgcgcagcgtaacttcattcttcaggtacagacagtgtttgtgt

SEQ ID NO: 311 - DNA, 75 bp, Artificial Sequence
  amplicon
cagtgcttccatggacaagtccttgtcaaaactggcccatgctgatggagatcaaacatcaatcatccctgtcca

SEQ ID NO: 312 - DNA, 84 bp, Artificial Sequence
  amplicon
ctctgagacagtgcttcgatgactttgcagacttggtgccctttgactcctgggagccgctcatgaggaagttgggcctcatgg

SEQ ID NO: 313 - DNA, 62 bp, Artificial Sequence
  amplicon
tgtcgatggacttccagaaccacctgggcagctgccaaaagtgtgatccaagctgtcccaat

SEQ ID NO: 314 - DNA, 67 bp, Artificial Sequence
  amplicon
aagccgcggttgaatgtgccatgaccctctccctctctggatggcaccatcattgaagctggcgtca

SEQ ID NO: 315 - DNA, 76 bp, Artificial Sequence
  amplicon
accgtaggctctgctctgaatgactctcctgtgggtctggctgcctatattctagagaagttttccacctggacca

SEQ ID NO: 316 - DNA, 81 bp, Artificial Sequence
  amplicon
cggttatgtcatgccagatacacacctcaaaggtactccctcctcccgggaaggcaccctttcttcagtgggtctcagttc

SEQ ID NO: 317 - DNA, 68 bp, Artificial Sequence
  amplicon
cgtggtgcccctctatgacctgctgctggagatgctggacgcccaccgcctacatgcgcccactagcc

SEQ ID NO: 318 - DNA, 74 bp, Artificial Sequence
  amplicon
cacgggacattcaccacatcgactactataaaaagacaaccaacggccgactgcctgtgaagtggatggcaccc

SEQ ID NO: 319 - DNA, 73 bp, Artificial Sequence
  amplicon
gcgtatgatttcccgaatgagtcaaaatgttgatatgcccaaacttcatgatgcaatgggtacgaggtcactg

SEQ ID NO: 320 - DNA, 68 bp, Artificial Sequence
  amplicon
tcagcagcaagggcatcatggaggaggatgaggcctgcgggcgccagtacacgctcaagaaaaccacc

SEQ ID NO: 321 - DNA, 75 bp, Artificial Sequence
  amplicon
caaaggagctcactgtggtgtctgtgttccaaccactgaatctggaccccatctgtgaataagccattctgactc

SEQ ID NO: 322 - DNA, 85 bp, Artificial Sequence
  amplicon
cttctgctaagcggtacatcgagacggacccagccaatcgggatcggcggacgcccatcaccgtggtgaagcaaggctttgagcc

SEQ ID NO: 323 - DNA, 76 bp, Artificial Sequence
  amplicon
gagaccctgctgtcccagaaccagggaggcaagaccttcattgtgggagaccagatctccttcgctgactacaacc

SEQ ID NO: 324 - DNA, 70 bp, Artificial Sequence
  amplicon
cggtgtgagaagtgcagcaagccctgtgcccgagtgtgctatggtctgggcatggagcacttgcgagagg

SEQ ID NO: 325 - DNA, 82 bp, Artificial Sequence
  amplicon
tgaacataaagtctgcaacatggaaggtattgcactgcacaggccacattcacgtatatgataccaacagtaaccaacctca

SEQ ID NO: 326 - DNA, 73 bp, Artificial Sequence
  amplicon
tccatgatggttctgcaggtttctgcggccccccggacagtggctctgacggcgttactgatggtgctgctca

SEQ ID NO: 327 - DNA, 73 bp, Artificial Sequence
  amplicon
tccaggatgttaggaactgtgaagatggaagggcatgaaaccagcgactggaacagctactacgcagacacgc

SEQ ID NO: 328 - DNA, 70 bp, Artificial Sequence
  amplicon
agaaccgcaaggtgagcaaggtggagattctccagcacgtcatcgactacatcagggaccttcagttgga

SEQ ID NO: 329 - DNA, 76 bp, Artificial Sequence
  amplicon
aacgactgctactccaagctcaaggagctggtgcccagcatcccccagaacaagaaggtgagcaagatggaaatcc

SEQ ID NO: 330 - DNA, 83 bp, Artificial Sequence
  amplicon
gcatggtagccgaagatttcacagtcaaaatcggagattttggtatgacgcgagatatctatgagacagactattaccggaaa

SEQ ID NO: 331 - DNA, 73 bp, Artificial Sequence
  amplicon
gtggacagcaccatgaacatgttgggcgggggaggcagtgctggccggaagcccctcaagtcgggtatgaagg

SEQ ID NO: 332 - DNA, 74 bp, Artificial Sequence
  amplicon
ccacagctcaccttctgtcaggtgtccatcccagctccagccagctcccagagaggaagagactggcactgagg

SEQ ID NO: 333 - DNA, 80 bp, Artificial Sequence
  amplicon
cggactttgggtgcgacttgacgagcggtggttcgacaagtggccttgcgggccggatcgtcccagtggaagagttgtaa

SEQ ID NO: 334 - DNA, 71 bp, Artificial Sequence
  amplicon
gcctagcagttctaccatgatcagcgtgcttcgagcgggtggagctctcagaaacatctggaccgatcacc

SEQ ID NO: 335 - DNA, 78 bp, Artificial Sequence
  amplicon
gcccagaggctccatcgtccatcctcttcctccccagtcggctgaactctccccttgtctgcactgttcaaacctctg

SEQ ID NO: 336 - DNA, 83 bp, Artificial Sequence
  amplicon
ggcctgctgagatcaaagactacagtccctacttcaagaccattgaggacctgaggaacaagattctcacagccacagtggac

SEQ ID NO: 337 - DNA, 73 bp, Artificial Sequence
  amplicon
cgaggattggttcttcagcaagacagaggaactgaaccgcgaggtggccaccaacagtgagctggtgcagagt

SEQ ID NO: 338 - DNA, 68 bp, Artificial Sequence
  amplicon
agagatcgaggctctcaaggaggagctgctcttcatgaagaagaaccacgaagaggaagtaaaaggcc

SEQ ID NO: 339 - DNA, 77 bp, Artificial Sequence
  amplicon
tgagcggcagaatcaggagtaccagcggctcatggacatcaagtcgcggctggagcaggagattgccacctaccgca

SEQ ID NO: 340 - DNA, 69 bp, Artificial Sequence
  amplicon
tcagtggagaaggagttggaccagtcaacatctctgttgtcacaagcagtgtttcctctggatatggca

SEQ ID NO: 341 - DNA, 75 bp, Artificial Sequence
  amplicon
gacttttgcccgctacctttcattccggcgtgacaacaatgagctgttgctcttcatactgaagcagttagtggc

SEQ ID NO: 342 - DNA, 75 bp, Artificial Sequence
  amplicon
ggagaacaatccccttgagacagaatatggcctttctgtctacaaggatcaccagaccatcaccatccaggagat

SEQ ID NO: 343 - DNA, 68 bp, Artificial Sequence
  amplicon
ctacagggacgccatcgaatccggatcttgatgctggtgtaagtgaacattcaggtgattggttggat

SEQ ID NO: 344 - DNA, 86 bp, Artificial Sequence
  amplicon
ccatgatggagaggcagacatcatgatcaactttggccgctgggagcatggcgatggatacccctttgacggtaaggacggactcc

SEQ ID NO: 345 - DNA, 67 bp, Artificial Sequence
  amplicon
gagaaccaatctcaccgacaggcagctggcagaggaatacctgtaccgctatggttacactcgggtg

SEQ ID NO: 346 - DNA, 75 bp, Artificial Sequence
  amplicon
acgagaacgagggcatctatgtgcaggatgtcaagaccggaaaggtgcgcgctgtgattggaagcacctacatgc

SEQ ID NO: 347 - DNA, 85 bp, Artificial Sequence
  amplicon
cggtacttctcagggctaatatatacgtactctggcctcttctgcgtggtggtcaacccctataaacacctgcccatctactcgg

SEQ ID NO: 348 - DNA, 79 bp, Artificial Sequence
  amplicon
gtgaggcagcgcgactctggcgactggccggccatgccttcccgggctgaggactatgaagtgttgtacaccattggca

SEQ ID NO: 349 - DNA, 68 bp, Artificial Sequence
  amplicon
ctgccgggatggcttctatgaggctgagctctgcccggaccgctgcatccacagtttccagaacctgg

SEQ ID NO: 350 - DNA, 73 bp, Artificial Sequence
  amplicon
ggctgtggctgaggctgtagcatctctgctggaggtgagacactctgggaactgatttgacctcgaatgctcc

SEQ ID NO: 351 - DNA, 66 bp, Artificial Sequence
  amplicon
ccagctctccttccagctacagatcaatgtccctgtccgagtgctggagctaagtgagagccaccc

SEQ ID NO: 352 - DNA, 76 bp, Artificial Sequence
  amplicon
cccatggatgctcctctgaagagactttcctcattgactgccgaggccccatgaatcaatgtctggtagccaccgg

SEQ ID NO: 353 - DNA, 85 bp, Artificial Sequence
  amplicon
gcatcaggctgtcattatggtgtccttacctgtgggagctgtaaggtcttctttaagagggcaatggaagggcagcacaactact

SEQ ID NO: 354 - DNA, 86 bp, Artificial Sequence
  amplicon
gccctcccagtgtgcaaataagggctgctgtttcgacgacaccgttcgtggggtcccctggtgcttctatcctaataccatcgacg

SEQ ID NO: 355 - DNA, 65 bp, Artificial Sequence
  amplicon
gggacactgcgggacaagagcggttccggagtctcaccactgcatttttcagagacgccatgggc

SEQ ID NO: 356 - DNA, 67 bp, Artificial Sequence
  amplicon
agctagcctcagtgacacacatgacaggttgcactgccgacgttgtgtcaacagccgtcagatccgg

SEQ ID NO: 357 - DNA, 77 bp, Artificial Sequence
  amplicon
cgaagcccttacaagtttcctagttcacccttacggattcctggagggaacatctatatttcacccctgaagagtcc

SEQ ID NO: 358 - DNA, 74 bp, Artificial Sequence
  amplicon
ccagacgagcgattagaagcggcagcttgtgaggtgaatgatttgggggaagaggaggaggaggaagaggagga

SEQ ID NO: 359 - DNA, 81 bp, Artificial Sequence
  amplicon
gctcattatgaaaaacatcccaaactttaaaatgcgaaattattggttggtgtgaagaaagccagacaacttctgtttctt

SEQ ID NO: 360 - DNA, 69 bp, Artificial Sequence
  amplicon
catcttccaggaggaccactctctgtggcaccctggactacctgccccctgaaatgattgaaggtcgga

SEQ ID NO: 361 - DNA, 90 bp, Artificial Sequence
  amplicon
cctggaggctgcaacatacctcaatcctgtcccaggccggatcctcctgaagcccttttcgcagcactgctatcctccaaagccattgta

SEQ ID NO: 362 - DNA, 80 bp, Artificial Sequence
  amplicon
tgttttgattcccgggcttaccaggtgagaagtgagggaggaagaaggcagtgtcccttttgctagagctgacagctttg

SEQ ID NO: 363 - DNA, 65 bp, Artificial Sequence
  amplicon
ggatcgagctcttccagatccttcggccagatgagcacattgccaaacagcgctatatcggtggc

SEQ ID NO: 364 - DNA, 69 bp, Artificial Sequence
  amplicon
tcaccctctgtgacttcatcgtgccctgggacaccctgagcaccacccagaagaagagcctgaaccaca

SEQ ID NO: 365 - DNA, 67 bp, Artificial Sequence
  amplicon
ctacctgccttgctttgtgacttccaagaacgagtgtctctggaccgacatgctctccaatttcggt

SEQ ID NO: 366 - DNA, 72 bp, Artificial Sequence
  amplicon
aatccaagggggagagtgatgacttccatatggactttgactcagctgtggctcctcgggcaaaatctgtac

SEQ ID NO: 367 - DNA, 74 bp, Artificial Sequence
  amplicon
tgctgttgctgagtctgttgccagtccccagaagaccatgtctgtgttgagctgtatctgtgaagccaggcaag

SEQ ID NO: 368 - DNA, 71 bp, Artificial Sequence
  amplicon
ctgctgtcttgggtgcattggagccttgccttgctgctctacctccaccatgccaagtggtcccaggctgc

SEQ ID NO: 369 - DNA, 71 bp, Artificial Sequence
  amplicon
tgacgatggcctggagtgtgtgcccactgggcagcaccaagtccggatgcagatcctcatgatccggtacc

SEQ ID NO: 370 - DNA, 83 bp, Artificial Sequence
  amplicon
cctcagcaagacgttatttgaaattacagtgcctctctctcaaggccccaaaccagtaacaatcagttttgccaatcacactt

SEQ ID NO: 371 - DNA, 72 bp, Artificial Sequence
  amplicon
tgcccttaaaggaaccaatgagtccctggaacgccagatgcgtgaaatggaagagaactttgccgttgaagc

SEQ ID NO: 372 - DNA, 70 bp, Artificial Sequence
  amplicon
acccagtagcaaggagaagcccactcactgctccgagtgcggcaaagctttcagaacctaccaccagctg

(19) United States
(12) Patent Application Publication
(10) Pub. No.: US 2005/0064455 A1
(43) Pub. Date: Mar. 24, 2005

(54) Gene expression markers for predicting response to chemotherapy

(76) Inventors: Joffre B. Baker, Montara, CA (US); Kathy D. Miller, Zionville, IN (US); Steven Shak, Hillsborough, CA (US); George W. Sledge, Indianapolis, IN (US); Sharon E. Soule, Lawrence, KS (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/852,797

(22) Filed: May 24, 2004

Related U.S. Application Data
(60) Provisional application No. 60/473,970, filed on May 28, 2003.

(51) Int. Cl. C12Q 1/68

(52) U.S. Cl. 435/6

(57) ABSTRACT

The invention provides sets of genes the expression of which predicts whether cancer patients are likely to have a beneficial treatment response to chemotherapy.

The present application claims the benefit under 35 U.S.C. 119(e) of the filing date of U.S. Application Ser. No. 60/473,970 filed on May 28, 2003.

### BACKGROUND OF THE INVENTION

1. Field of the Invention

The present invention provides sets of genes the expression of which is important in the prognosis of cancer. In particular, the invention provides gene expression information useful for predicting whether cancer patients are likely to have a beneficial treatment response to chemotherapy.

2. Description of the Related Art

Oncologists have a number of treatment options available to them, including different combinations of chemotherapeutic drugs that are characterized as “standard of care,” and a number of drugs that do not carry a label claim for particular cancer, but for which there is evidence of efficacy in that cancer. Best likelihood of good treatment outcome requires that patients be assigned to optimal available cancer treatment, and that this assignment be made as quickly as possible following diagnosis. In particular, it is important to determine the likelihood of patient response to “standard of care” chemotherapy because chemotherapeutic drugs such as anthracyclines and taxanes have limited efficacy and are toxic. The identification of patients who are most or least likely to respond thus could increase the net benefit these drugs have to offer, and decrease the net morbidity and toxicity, via more intelligent patient selection.

Currently, diagnostic tests used in clinical practice are single analyte, and therefore do not capture the potential value of knowing relationships between dozens of different markers. Moreover, diagnostic tests are frequently not quantitative, relying on immunohistochemistry. This method often yields different results in different laboratories, in part because the reagents are not standardized, and in part because the interpretations are subjective and cannot be easily quantified. RNA-based tests have not often been used because of the problem of RNA degradation over time and the fact that it is difficult to obtain fresh tissue samples from patients for analysis. Fixed paraffin-embedded tissue is more readily available and methods have been established to detect RNA in fixed tissue. However, these methods typically do not allow for the study of large numbers of genes (DNA or RNA) from small amounts of material. Thus, traditionally fixed tissue has been rarely used other than for immunohistochemistry detection of proteins.

Recently, several groups have published studies concerning the classification of various cancer types by microarray gene expression analysis (see, e.g. Golub et al., Science 286:531-537 (1999); Bhattacharjae et al., Proc. Natl. Acad. Sci. USA 98:13790-13795 (2001); Chen-Hsiang et al., Bioinformatics 17 (Suppl. 1):S316-S322 (2001); Ramaswamy et al., Proc. Natl. Acad. Sci. USA 98:15149-15154 (2001)). Certain classifications of human breast cancers based on gene expression patterns have also been reported (Martin et al., Cancer Res. 60:2232-2238 (2000); West et al., Proc. Natl. Acad. Sci. USA 98:11462-11467 (2001); Sorlie et al., Proc. Natl. Acad. Sci. USA 98:10869-10874 (2001); Yan et al., Cancer Res. 61:8375-8380 (2001)). However, these studies mostly focus on improving and refining the already established classification of various types of cancer, including breast cancer, and generally do not provide new insights into the relationships of the differentially expressed genes, and do not link the findings to treatment strategies in order to improve the clinical outcome of cancer therapy.

Although modern molecular biology and biochemistry have revealed hundreds of genes whose activities influence the behavior of tumor cells, state of their differentiation, and their sensitivity or resistance to certain therapeutic drugs, with a few exceptions, the status of these genes has not been exploited for the purpose of routinely making clinical decisions about drug treatments. One notable exception is the use of estrogen receptor (ER) protein expression in breast carcinomas to select patients to treatment with anti-estrogen drugs, such as tamoxifen. Another exceptional example is the use of ErbB2 (Her2) protein expression in breast carcinomas to select patients with the Her2 antagonist drug Herceptin® (Genentech, Inc., South San Francisco, Calif.).

Despite recent advances, the challenge of cancer treatment remains to target specific treatment regimens to pathogenically distinct tumor types, and ultimately personalize tumor treatment in order to maximize outcome. Hence, a need exists for tests that simultaneously provide predictive information about patient responses to the variety of treatment options. This is particularly true for breast cancer, the biology of which is poorly understood. It is clear that the classification of breast cancer into a few subgroups, such as the ErbB2 positive subgroup, and subgroups characterized by low to absent gene expression of the estrogen receptor (ER) and a few additional transcriptional factors (Perou et al., Nature 406:747-752 (2000)), does not reflect the cellular and molecular heterogeneity of breast cancer, and does not allow the design of treatment strategies maximizing patient response.

Breast cancer is the most common type of cancer among women in the United States and is the leading cause of cancer deaths among women ages 40-59. Therefore, there is a particularly great need for a clinically validated breast cancer test predictive of patient response to chemotherapy.

### SUMMARY OF THE INVENTION

The present invention provides gene sets useful in predicting the response of cancer, e.g. breast cancer patients to chemotherapy. In addition, the invention provides a clinically validated cancer, e.g. breast cancer, test predictive of patient response to chemotherapy, using multi-gene RNA analysis. The present invention accommodates the use of archived paraffin-embedded biopsy material for assay of all markers in the relevant gene sets, and therefore is compatible with the most widely available type of biopsy material.

In one aspect, the invention concerns a method for predicting the response of a subject diagnosed with cancer to chemotherapy comprising determining the expression level of one or more prognostic RNA transcripts or their expression products in a biological sample comprising cancer cells obtained from said subject, wherein the prognostic RNA transcript is the transcript of one or more genes selected from the group consisting of VEGFC; B-Catenin; MMP2; MMP9; CNN; FLJ20354; TGFB3; PDGFRb; PLAUR; KRT19; ID1; RIZ1; RAD54L; RB1; SURV; EIF4EL3; CYP2C8; STK15; ACTG2; NEK2; cMet; TIMP2; C20 orf1; DR5; CD31; BIN1; COL1A2; HIF1A; VIM; CDC20; ID2; MCM2; CCNB1; MYH11; Chk2; G-Catenin; HER2; GSN; Ki-67; TOP2A; CCND1; EstR1; KRT18; GATA3; cIAP2; KRT5; RAB27B; IGF1R; HNF3A; CA9; MCM3; STMY3; NPD009; BAD; BBC3; EGFR; CD9; AKT1; CD3z; KRT14; DKFZp564; Bcl2; BECN1; KLK10; DIABLO; MVP; VEGFB; ErbB3; MDM2; Bclx; CDH1; HLA-DPB1; PR; KRT17; GSTp; IRS1; NFKBp65; IGFBP2; RPS6 KB1; DHPS; TIMP3; ZNF217; KIAA1209; COX2; pS2; BRK; CEGP1; EPHX1; VEGF; TP53BP1; COL1A1; FGFR1; and CTSL2, wherein
(a) for every unit of increased expression of one or more of MMP9; FLJ20354; RAD54L; SURV; CYP2C8; STK15; NEK2; C20 orf1; CDC20; MCM2; CCNB1; Chk2; Ki-67; TOP2A; CCND1; EstR1; KRT18; GATA3; RAB27B; IGF1R; HNF3A; STMY3; NPD009; BAD; BBC3; CD9; AKT1; Bcl2; BECN1; DIABLO; MVP; VEGFB; ErbB3; MDM2; Bclx; CDH1; PR; IRS1; NFKBp65; IGFBP2; RPS6 KB1; DHPS; TIMP3; ZNF217; pS2; BRK; CEGP1; EPHX1; TP53BP1; COL1A1; and FGFR1, or the corresponding expression product, the subject is predicted to have an increased likelihood of response; and
(b) for every unit of increased expression of one or more of VEGFC; B-Catenin; MMP2; CNN; TGFB3; PDGFRb; PLAUR; KRT19; ID1; RIZ1; RB1; EIF4EL3; ACTG2; cMet; TIMP2; DR5; CD31; BIN1; COL1A2; HIF1A; VIM; ID2; MYH11; G-Catenin; HER2; GSN; cIAP2; KRT5; CA9; MCM3; EGFR; CD3z; KRT14; DKFZp564; KLK10; HLA-DPB1; KRT17; GSTp; KIAA1209; COX2; VEGF; and CTSL2, or the corresponding expression product, the subject is predicted to have a decreased likelihood of response.

In a particular embodiment, response is clinical response, the prognostic RNA transcript is the transcript of one or more genes selected from the group consisting of CCND1; EstR1; KRT18; GATA3; cIAP2; KRT5; RAB27B; IGF1R; HNF3A; CA9; MCM3; STMY3; NPD009; BAD; BBC3; EGFR; CD9; AKT1; CD3z; KRT14; DKFZp564; Bcl2; BECN1; KLK10; DIABLO; MVP; VEGFB; ErbB3; MDM2; Bclx; CDH1; HLA-DPB1; PR; KRT17; GSTp; IRS1; NFKBp65; IGFBP2; RPS6 KB1; DHPS; TIMP3; ZNF217; KIAA1209; COX2; pS2; BRK; CEGP1; EPHX1; VEGF; TP53BP1; COL1A1; FGFR1; and CTSL2; and
(a) for every unit of increased expression of one or more of CCND1; EstR1; KRT18; GATA3; RAB27B; IGF1R; HNF3A; STMY3; NPD009; BAD; BBC3; CD9; AKT1; Bcl2; BECN1; DIABLO; MVP; VEGFB; ErbB3; MDM2; Bclx; CDH1; PR; IRS1; NFKBp65; IGFBP2; RPS6 KB1; DHPS; TIMP3; ZNF217; pS2; BRK; CEGP1; EPHX1; TP53BP1; COL1A1; and FGFR1, or the corresponding expression products the subject is predicted to have an increased likelihood of clinical response; and
(b) for every unit of increased expression of one or more of cIAP2; KRT5; CA9; MCM3; EGFR; CD3z; KRT14; DKFZp564; KLK10; HLA-DPB1; KRT17; GSTp; KIAA1209; COX2; VEGF; and CTSL2, or the corresponding expression products the subject is predicted to have a decreased likelihood of clinical response.

In another embodiment, the response is a pathogenic response, the prognostic RNA transcript is the transcript of one or more genes selected from the group consisting of VEGFC; B-Catenin; MMP2; MMP9; CNN; FLJ20354; TGFB3; PDGFRb; PLAUR; KRT19; ID1; RIZ1; RAD54L; RB1; SURV; EIF4EL3; CYP2C8; STK15; ACTG2; NEK2; cMet; TIMP2; C20 orf1; DR5; CD31; BIN1; COL1A2; HIF1A; VIM; CDC20; ID2; MCM2; CCNB1; MYH11; Chk2; G-Catenin; HER2; GSN; Ki-67; TOP2A; and
(a) for every unit of increased expression of one or more of MMP9; FLJ20354; RAD54L; SURV; CYP2C8; STK15; NEK2; C20 orf1; CDC20; MCM2; CCNB1; Chk2; Ki-67; TOP2A, or the corresponding expression products the subject is predicted to have an increased likelihood of pathological response; and
(b) for every unit of increased expression of one or more of VEGFC; B-Catenin; MMP2; CNN; TGFB3; PDGFRb; PLAUR; KRT19; ID1; RIZ1; RB1; EIF4EL3; ACTG2; cMet; TIMP2; DR5; CD31; BIN1; COL1A2; HIF1A; VIM; ID2; MYH11; G-Catenin; HER2; GSN, or the corresponding expression products the subject is predicted to have a decreased likelihood of pathological response.

In a particular embodiment of this method, the expression level of at least 2, or at least 5, or at least 10, or at lest 15 predictive RNA transcripts or their expression products is determined.

In another embodiment, RNA is obtained from a fixed, paraffin-embedded cancer tissue specimen of the subject. The subject preferably is a human patient.

The cancer can be any kind of cancer, including, for example, breast cancer, ovarian cancer, gastric cancer, colorectal cancer, pancreatic cancer, prostate cancer, and lung cancer, in particular, breast cancer, such as invasive breast cancer.

In another aspect, the invention concerns an array comprising polynucleotides hybridizing to one or more of the following genes: VEGFC; B-Catenin; MMP2; MMP9; CNN; FLJ20354; TGFB3; PDGFRb; PLAUR; KRT19; ID1; RIZ1; RAD54L; RB1; SURV; EIF4EL3; CYP2C8; STK15; ACTG2; NEK2; cMet; TIMP2; C20 orf1; DR5; CD31; BIN1; COL1A2; HIF1A; VIM; CDC20; ID2; MCM2; CCNB1; MYH11; Chk2; G-Catenin; HER2; GSN; Ki-67; TOP2A; CCND1; EstR1; KRT18; GATA3; cIAP2; KRT5; RAB27B; IGF1R; HNF3A; CA9; MCM3; STMY3; NPD009; BAD; BBC3; EGFR; CD9; AKT1; CD3z; KRT14; DKFZp564; Bcl2; BECN1; KLK10; DIABLO; MVP; VEGFB; ErbB3; MDM2; Bclx; CDH1; HLA-DPB1; PR; KRT17; GSTp; IRS1; NFKBp65; IGFBP2; RPS6 KB1; DHPS; TIMP3; ZNF217; KIAA1209; COX2; pS2; BRK; CEGP1; EPHX1; VEGF; TP53BP1; COL1A1; FGFR1; and CTSL2, immobilized on a solid surface.

In yet another aspect, the invention concerns an array comprising polynucleotides hybridizing to one or more of the following genes: CCND1; EstR1; KRT18; GATA3; cIAP2; KRT5; RAB27B; IGF1R; HNF3A; CA9; MCM3; STMY3; NPD009; BAD; BBC3; EGFR; CD9; AKT1; CD3z; KRT14; DKFZp564; Bcl2; BECN1; KLK10; DIABLO; MVP; VEGFB; ErbB3; MDM2; Bclx; CDH1; HLA-DPB1; PR; KRT17; GSTp; IRS1; NFKBp65; IGFBP2; RPS6 KB1; DHPS; TIMP3; ZNF217; KIAA1209; COX2; pS2; BRK; CEGP1; EPHX1; VEGF; TP53BP1; COL1A1; FGFR1; and CTSL2, immobilized on a solid surface.

In a further embodiment, the invention concerns an array comprising polynucleotides hybridizing to one or more of the following genes: VEGFC; B-Catenin; MMP2; MMP9; CNN; FLJ20354; TGFB3; PDGFRb; PLAUR; KRT19; ID1; RIZ1; RAD54L; RB1; SURV; EIF4EL3; CYP2C8; STK15; ACTG2; NEK2; cMet; TIMP2; C20 orf1; DR5; CD31; BIN1; COL1A2; HIF1A; VIM; CDC20; ID2; MCM2; CCNB1; MYH11; Chk2; G-Catenin; HER2; GSN; Ki-67; TOP2A, immobilized on a solid surface.

In all embodiments, the array might contain a plurality of polynucleotides, hybridizing to the listed genes, where “plurality” means any number more than one. The polynucleotides might include intron-based sequences, the expression of which correlates with the expression of the corresponding exon.

In all aspects, the polynucleotides can be cDNAs (“cDNA arrays) that are typically about 500 to 5000 bases long, although shorter or longer cDNAs can also be used and are within the scope of this invention. Alternatively, the polynucleotides can be oligonucleotides (DNA microarrays), which are typically about 20 to 80 bases long, although shorter and longer oligonucleotides are also suitable and are within the scope of the invention. The solid surface can, for example, be glass or nylon, or any other solid surface typically used in preparing arrays, such as microarrays, and is typically glass. Hybridization typically conducted under stringent conditions, or moderately stringent conditions. In various embodiments, the array comprises polynucleotides hybridizing to at least two, at least three, at least four, at least five, at least six, at least seven, etc. of the genes listed above. Hybridization to any number of genes selected from the genes present on the arrays, in any combination is included.

In another aspect, the invention concerns a method of preparing a personalized genomics profile for a patient comprising the steps of:
(a) subjecting RNA extracted from cancer cells obtained from said patient to gene expression analysis;
(b) determining the expression level of at least one gene selected from the group consisting of VEGFC; B-Catenin; MMP2; MMP9; CNN; FLJ20354; TGFB3; PDGFRb; PLAUR; KRT19; ID1; RIZ1; RAD54L; RB1; SURV; EIF4EL3; CYP2C8; STK15; ACTG2; NEK2; cMet; TIMP2; C20 orf1; DR5; CD31; BIN1; COL1A2; HIF1A; VIM; CDC20; ID2; MCM2; CCNB1; MYH11; Chk2; G-Catenin; HER2; GSN; Ki-67; TOP2A; CCND1; EstR1; KRT18; GATA3; cIAP2; KRT5; RAB27B; IGF1R; HNF3A; CA9; MCM3; STMY3; NPD009; BAD; BBC3; EGFR; CD9; AKT1; CD3z; KRT14; DKFZp564; Bcl2; BECN1; KLK10; DIABLO; MVP; VEGFB; ErbB3; MDM2; Bclx; CDH1; HLA-DPB1; PR; KRT17; GSTp; IRS1; NFKBp65; IGFBP2; RPS6 KB1; DHPS; TIMP3; ZNF217; KIAA1209; COX2; pS2; BRK; CEGP1; EPHX1; VEGF; TP53BP1; COL1A1; FGFR1; and CTSL2; wherein the expression level is normalized against a control gene or genes and optionally is compared to the amount found in a corresponding cancer reference tissue set; and
(c) creating a report summarizing the data obtained by said gene expression analysis.

The breast tissue may contain breast cancer cells, and the RNA may be obtained from a dissected portion of the tissue enriched for such breast cancer cells. As a control gene, any known reference gene can be used, including, for example, glyceraldehyde-3-phosphate dehydrogenase (GAPDH), β-actin, U-snRNP-associated cyclophilin (USA-CYP), and ribosomal protein LPO. Alternatively, normalization can be achieved by correcting for differences between the total of all signals of the tested gene sets (global normalization strategy). The report may include a prognosis for the outcome of the treatment of the patient. The method may additionally comprise the step of treating the subject, e.g. a human patient, if a good prognosis is indicated.

In an additional aspect, the invention concerns a PCR primer-probe set listed in Table 3, and a PCR amplicon listed in Table 4.

### BRIEF DESCRIPTION OF THE DRAWINGS

Table 1 is a list of genes, expression of which correlate, positively or negatively, with breast cancer response to adriamycin and taxane chemotherapy. Results from a retrospective clinical trial. Binary statistical analysis with pathological response endpoint.

Table 2 is a list of genes, expression of which correlate, positively or negatively, with breast cancer response to adriamycin and taxane chemotherapy. Results from a retrospective clinical trial. Binary statistical analysis with clinical response endpoint.

Table 3 is a list of genes, expression of which predict breast cancer response to chemotherapy. Results from a retrospective clinical trial. The table includes accession numbers for the genes, and sequences for the forward and reverse primers (designated by “f” and “r”, respectively) and probes (designated by “p”) used for PCR amplification.

Table 4 shows the amplicon sequences used in PCR amplification of the indicated genes.

### DETAILED DESCRIPTION

A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “microarray” refers to an ordered arrangement of hybridizable array elements, preferably polynucleotide probes, on a substrate.

The term “polynucleotide,” when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The terms “differentially expressed gene,” “differential gene expression” and their synonyms, which are used interchangeably, refer to a gene whose expression is activated to a higher or lower level in a subject suffering from a disease, specifically cancer, such as breast cancer, relative to its expression in a normal or control subject. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disease, specifically cancer, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages. For the purpose of this invention, “differential gene expression” is considered to be present when there is at least an about two-fold, preferably at least about four-fold, more preferably at least about six-fold, most preferably at least about ten-fold difference between the expression of a given gene in normal and diseased subjects, or in various stages of disease development in a diseased subject.

The phrase “gene amplification” refers to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

The term “over-expression” with regard to an RNA transcript is used to refer the level of the transcript determined by normalization to the level of reference mRNAs, which might be all measured transcripts in the specimen or a particular reference set of mRNAs.

The term “prognosis” is used herein to refer to the prediction of the likelihood of cancer-attributable death or progression, including recurrence, metastatic spread, and drug resistance, of a neoplastic disease, such as breast cancer. The term “prediction” is used herein to refer to the likelihood that a patient will respond either favorably or unfavorably to a drug or set of drugs, and also the extent of those responses, or that a patient will survive, following surgical removal or the primary tumor and/or chemotherapy for a certain period of time without cancer recurrence. The predictive methods of the present invention can be used clinically to make treatment decisions by choosing the most appropriate treatment modalities for any particular patient. The predictive methods of the present invention are valuable tools in predicting if a patient is likely to respond favorably to a treatment regimen, such as surgical intervention, chemotherapy with a given drug or drug combination, and/or radiation therapy, or whether long-term survival of the patient, following surgery and/or termination of chemotherapy or other treatment modalities is likely.

The term “long-term” survival is used herein to refer to survival for at least 3 years, more preferably for at least 8 years, most preferably for at least 10 years following surgery or other treatment.

The term “tumor,” as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include but are not limited to, breast cancer, colorectal cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

“Patient response” can be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition (i.e. reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

The term “(lymph) node negative” cancer, such as “(lymph) node negative” breast cancer, is used herein to refer to cancer that has not spread to the lymph nodes.

The term “gene expression profiling” is used in the broadest sense, and includes methods of quantification of mRNA and/or protein levels in a biological sample.

“Neoadjuvant therapy” is adjunctive or adjuvant therapy given prior to the primary (main) therapy. Neoadjuvant therapy includes, for example, chemotherapy, radiation therapy, and hormone therapy. Thus, chemotherapy may be administered prior to surgery to shrink the tumor, so that surgery can be more effective, or, in the case of previously inoperable tumors, possible.

The term “cancer-related biological function” is used herein to refer to a molecular activity that impacts cancer success against the host, including, without limitation, activities regulating cell proliferation, programmed cell death (apoptosis), differentiation, invasion, metastasis, tumor suppression, susceptibility to immune surveillance, angiogenesis, maintenance or acquisition of immortality.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, typically: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

In the context of the present invention, reference to “at least one,” “at least two,” “at least five,” etc. of the genes listed in any particular gene set means any one or any and all combinations of the genes listed.

The term “normalized” with regard to a gene transcript or a gene expression product refers to the level of the transcript or gene expression product relative to the mean levels of transcripts/products of a set of reference genes, wherein the reference genes are either selected based on their minimal variation across, patients, tissues or treatments (“housekeeping genes”), or the reference genes are the totality of tested genes. In the latter case, which is commonly referred to as “global normalization”, it is important that the total number of tested genes be relatively large, preferably greater than 50. Specifically, the term ‘normalized’ with respect to an RNA transcript refers to the transcript level relative to the mean of transcript levels of a set of reference genes. More specifically, the mean level of an RNA transcript as measured by TaqMan® RT-PCR refers to the Ct value minus the mean Ct values of a set of reference gene transcripts.

The terms “expression threshold,” and “defined expression threshold” are used interchangeably and refer to the level of a gene or gene product in question above which the gene or gene product serves as a predictive marker for patient response or resistance to a drug. The threshold typically is defined experimentally from clinical studies. The expression threshold can be selected either for maximum sensitivity (for example, to detect all responders to a drug), or for maximum selectivity (for example to detect only responders to a drug), or for minimum error.

B. Detailed Description

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

1. Gene Expression Profiling

Methods of gene expression profiling include methods based on hybridization analysis of polynucleotides, methods based on sequencing of polynucleotides, and proteomics-based methods. The most commonly used methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and PCR-based methods, such as reverse transcription polymerase chain reaction (RT-PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

2. PCR-Based Gene Expression Profiling Methods

a. Reverse Transcriptase PCR (RT-PCR)

One of the most sensitive and most flexible quantitative PCR-based gene expression profiling methods is RT-PCR, which can be used to compare mRNA levels in different sample populations, in normal and tumor tissues, with or without drug treatment, to characterize patterns of gene expression, to discriminate between closely related mRNAs, and to analyze RNA structure.

The first step is the isolation of mRNA from a target sample. The starting material is typically total RNA isolated from human tumors or tumor cell lines, and corresponding normal tissues or cell lines, respectively. Thus RNA can be isolated from a variety of primary tumors, including breast, lung, colorectal, prostate, brain, liver, kidney, pancreas, spleen, thymus, testis, ovary, uterus, etc., tumor, or tumor cell lines, with pooled DNA from healthy donors. If the source of mRNA is a primary tumor, mRNA can be extracted, for example, from frozen or archived paraffin-embedded and fixed (e.g. formalin-fixed) tissue samples.

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andrés et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, CA, USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Cₜ).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

b. MassARRAY System

In the MassARRAY-based gene expression profiling method, developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059-3064 (2003).

c. Other PCR-Based Methods

Further PCR-based techniques include, for example, differential display (Liang and Pardee, Science 257:967-971 (1992)); amplified fragment length polymorphism (iAFLP) (Kawamoto et al., Genome Res. 12:1305-1312 (1999)); BeadArray™ technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex¹⁰⁰ LabMAP system and multiple color-coded microspheres (Luminex Corp., Austin, Tex.) in a rapid assay for gene expression (Yang et al., Genome Res. 11:1888-1898 (2001)); and high coverage expression profiling (HiCEP) analysis (Fukumura et al., Nucl. Acids. Res. 31(16) e94 (2003)).

3. Microarrays

Differential gene expression can also be identified, or confirmed using the microarray technique. Thus, the expression profile of breast cancer-associated genes can be measured in either fresh or paraffin-embedded tumor tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from human tumors or tumor cell lines, and corresponding normal tissues or cell lines. Thus RNA can be isolated from a variety of primary tumors or tumor cell lines. If the source of mRNA is a primary tumor, mRNA can be extracted, for example, from frozen or archived paraffin-embedded and fixed (e.g. formalin-fixed) tissue samples, which are routinely prepared and preserved in everyday clinical practice.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106-149 (1996)). Microarray analysis can be performed by commercially available equipment, following manufacturer's protocols, such as by using the Affymetrix GenChip technology, or Incyte's microarray technology.

The development of microarray methods for large-scale analysis of gene expression makes it possible to search systematically for molecular markers of cancer classification and outcome prediction in a variety of tumor types.

4. Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al., Cell 88:243-51 (1997).

5. Gene Expression Analysis by Massively Parallel Signature Sequencing (MPSS)

This method, described by Brenner et al., Nature Biotechnology 18:630-634 (2000), is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

6. Immunohistochemistry

Immunohistochemistry methods are also suitable for detecting the expression levels of the prognostic markers of the present invention. Thus, antibodies or antisera, preferably polyclonal antisera, and most preferably monoclonal antibodies specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

7. Proteomics

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

8. General Description of mRNA Isolation, Purification and Amplification

The steps of a representative protocol for profiling gene expression using fixed, paraffin-embedded tissues as the RNA source, including mRNA isolation, purification, primer extension and amplification are given in various published journal articles (for example: T. E. Godfrey et al. J. Molec. Diagnostics 2: 84-91 [2000]; K. Specht et al., Am. J. Pathol. 158: 419-29 [2001]). Briefly, a representative process starts with cutting about 10 μm thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by RT-PCR. Finally, the data are analyzed to identify the best treatment option(s) available to the patient on the basis of the characteristic gene expression pattern identified in the tumor sample examined.

9. Cancer Chemotherapy

Chemotherapeutic agents used in cancer treatment can be divided into several groups, depending on their mechanism of action. Some chemotherapeutic agents directly damage DNA and RNA. By disrupting replication of the DNA such chemotherapeutics either completely halt replication, or result in the production of nonsense DNA or RNA. This category includes, for example, cisplatin (Platinol®), daunorubicin (Cerubidine®), doxorubicin (Adriamycin®), and etoposide (VePesid®). Another group of cancer chemotherapeutic agents interfere with the formation of nucleotides or deoxyribonucleotides, so that RNA synthesis and cell replication is blocked. Examples of drugs in this class include methotrexate (Abitrexate®), mercaptopurine (Purinethol®), fluorouracil (Adrucil®), and hydroxyurea (Hydrea®). A third class of chemotherapeutic agents effects the synthesis or breakdown of mitotic spindles, and, as a result, interrupt cell division. Examples of drugs in this class include Vinblastine (Velban®), Vincristine (Oncovin®) and taxenes, such as, Pacitaxel (Taxol®), and Tocetaxel (Taxotere®) Tocetaxel is currently approved in the United States to treat patients with locally advanced or metastatic breast cancer after failure of prior chemotherapy, and patients with locally advanced or metastatic non-small cell lung cancer after failure of prior platinum-based chemotherapy. The prediction of patient response to all of these, and other chemotherapeutic agents is specifically within the scope of the present invention.

In a specific embodiment, chemotherapy includes treatment with a taxane derivative. Taxanes include, without limitation, paclitaxel (Taxol®) and docetaxel (Taxotere®), which are widely used in the treatment of cancer. As discussed above, taxanes affect cell structures called microtubules, which play an important role in cell functions. In normal cell growth, microtubules are formed when a cell starts dividing. Once the cell stops dividing, the microtubules are broken down or destroyed. Taxanes stop the microtubules from breaking down; cancer cells become so clogged with microtubules that they cannot grow and divide.

In another specific embodiment, chemotherapy includes treatment with an anthracycline derivative, such as, for example, doxorubicin, daunorubicin, and aclacinomycin.

In a further specific embodiment, chemotherapy includes treatment with a topoisomerase inhibitor, such as, for example, camptothecin, topotecan, irinotecan, 20-S-camptothecin, 9-nitro-camptothecin, 9-amino-camptothecin, or GI147211.

Treatment with any combination of these and other chemotherapeutic drugs is specifically contemplated.

Most patients receive chemotherapy immediately following surgical removal of tumor. This approach is commonly referred to as adjuvant therapy. However, chemotherapy can be administered also before surgery, as so called neoadjuvant treatment. Although the use of neo-adjuvant chemotherapy originates from the treatment of advanced and inoperable breast cancer, it has gained acceptance in the treatment of other types of cancers as well. The efficacy of neoadjuvant chemotherapy has been tested in several clinical trials. In the multi-center National Surgical Adjuvant Breast and Bowel Project B-18 (NSAB B-18) trial (Fisher et al., J. Clin. Oncology 15:2002-2004 (1997); Fisher et al., J. Clin. Oncology 16:2672-2685 (1998)) neoadjuvant therapy was performed with a combination of adriamycin and cyclophosphamide (“AC regimen”). In another clinical trial, neoadjuvant therapy was administered using a combination of 5-fluorouracil, epirubicin and cyclophosphamide (“FEC regimen”) (van Der Hage et al., J. Clin. Oncol. 19:4224-4237 (2001)). Newer clinical trials have also used taxane-containing neoadjuvant treatment regiments. See, e.g. Holmes et al., J. Natl. Cancer Inst. 83:1797-1805 (1991) and Moliterni et al., Seminars in Oncology, 24:S17-10-S-17-14 (1999). For further information about neoadjuvant chemotherapy for breast cancer see, Cleator et al., Endocrine-Related Cancer 9:183-195 (2002).

10. Cancer Gene Set, Assayed Gene Subsequences, and Clinical Application of Gene Expression Data

An important aspect of the present invention is to use the measured expression of certain genes by breast cancer tissue to provide prognostic information. For this purpose it is necessary to correct for (normalize away) both differences in the amount of RNA assayed and variability in the quality of the RNA used. Therefore, the assay typically measures and incorporates the expression of certain normalizing genes, including well known housekeeping genes, such as GAPDH and Cyp1. Alternatively, normalization can be based on the mean or median signal (Ct) of all of the assayed genes or a large subset thereof (global normalization approach). On a gene-by-gene basis, measured normalized amount of a patient tumor mRNA is compared to the amount found in a breast cancer tissue reference set. The number (N) of breast cancer tissues in this reference set should be sufficiently high to ensure that different reference sets (as a whole) behave essentially the same way. If this condition is met, the identity of the individual breast cancer tissues present in a particular set will have no significant impact on the relative amounts of the genes assayed. Usually, the breast cancer tissue reference set consists of at least about 30, preferably at least about 40 different FPE breast cancer tissue specimens. Unless noted otherwise, normalized expression levels for each mRNA/tested tumor/patient will be expressed as a percentage of the expression level measured in the reference set. More specifically, the reference set of a sufficiently high number (e.g. 40) of tumors yields a distribution of normalized levels of each mRNA species. The level measured in a particular tumor sample to be analyzed falls at some percentile within this range, which can be determined by methods well known in the art. Below, unless noted otherwise, reference to expression levels of a gene assume normalized expression relative to the reference set although this is not always explicitly stated.

11. Recurrence Scores

Copending application Ser. No. 60/486,302 describes an algorithm-based prognostic test for determining the likelihood of cancer recurrence and/or the likelihood that a patient responds well to a treatment modality. Features of the algorithm that distinguish it from other cancer prognostic methods include: 1) a unique set of test mRNAs (or the corresponding gene expression products) used to determine recurrence likelihood, 2) certain weights used to combine the expression data into a formula, and 3) thresholds used to divide patients into groups of different levels of risk, such as low, medium, and high risk groups. The algorithm yields a numerical recurrence score (RS) or, if patient response to treatment is assessed, response to therapy score (RTS).

The test requires a laboratory assay to measure the levels of the specified mRNAs or their expression products, but can utilize very small amounts of either fresh tissue, or frozen tissue or fixed, paraffin-embedded tumor biopsy specimens that have already been necessarily collected from patients and archived. Thus, the test can be noninvasive. It is also compatible with several different methods of tumor tissue harvest, for example, via core biopsy or fine needle aspiration.

According to the method, cancer recurrence score (RS) is determined by:
(a) subjecting a biological sample comprising cancer cells obtained from said subject to gene or protein expression profiling;
(b) quantifying the expression level of multiple individual genes [i.e., levels of mRNAs or proteins] so as to determine an expression value for each gene;
(c) creating subsets of the gene expression values, each subset comprising expression values for genes linked by a cancer-related biological function and/or by co-expression;
(d) multiplying the expression level of each gene within a subset by a coefficient reflecting its relative contribution to cancer recurrence or response to therapy within said subset and adding the products of multiplication to yield a term for said subset;
(e) multiplying the term of each subset by a factor reflecting its contribution to cancer recurrence or response to therapy; and
(f) producing the sum of terms for each subset multiplied by said factor to produce a recurrence score (RS) or a response to therapy (RTS) score,
wherein the contribution of each subset which does not show a linear correlation with cancer recurrence or response to therapy is included only above a predetermined threshold level, and
wherein the subsets in which increased expression of the specified genes reduce risk of cancer recurrence are assigned a negative value, and the subsets in which expression of the specified genes increase risk of cancer recurrence are assigned a positive value.

In a particular embodiment, RS is determined by:
(a) determining the expression levels of GRB7, HER2, EstR1, PR, Bcl2, CEGP1, SURV, Ki.67, MYBL2, CCNB1, STK15, CTSL2, STMY3, CD68, GSTM1, and BAG1, or their expression products, in a biological sample containing tumor cells obtained from said subject; and
(b) calculating the recurrence score (RS) by the following equation:
   RS=(0.23 to 0.70)×GRB7axisthresh−(0.17 to 0.51)×ERaxis+(0.53 to 1.56)×prolifaxisthresh+(0.07 to 0.21)×invasionaxis+(0.03 to 0.15)×CD68−(0.04 to 0.25)×GSTM1−(0.05 to 0.22)×BAG1
wherein
   (i) GRB7 axis=(0.45 to 1.35)×GRB7+(0.05 to 0.15)×HER2;
   (ii) if GRB7 axis<−2, then GRB7 axis thresh=−2, and
   if GRB7 axis≧−2, then GRB7 axis thresh=GRB7 axis;
   (iii) ER axis=(Est1+PR+Bcl2+CEGP1)/4;
   (iv) prolifaxis=(SURV+Ki.67+MYBL2+CCNB1+STK15)/5;
   (v) if prolifaxis<−3.5, then prolifaxisthresh=−3.5,
   if prolifaxis≧−3.5, then prolifaxishresh=prolifaxis; and
   (vi) invasionaxis=(CTSL2+STMY3)/2,
wherein the terms for all individual genes for which ranges are not specifically shown can vary between about 0.5 and 1.5, and wherein a higher RS represents an increased likelihood of cancer recurrence.

Further details of the invention will be described in the following non-limiting Example.

### EXAMPLE

A Retrospective Study of Neoadjuvant Chemotherapy in Invasive Breast Cancer: Gene Expression Profiling of Paraffin-Embedded Core Biopsy Tissue

A gene expression study was designed and conducted with the primary goal to molecularly characterize gene expression in paraffin-embedded, fixed tissue samples of invasive breast ductal carcinoma, and to explore the correlation between such molecular profiles and patient response to chemotherapy.

Study Design

70 Patients with newly diagnosed stage II or stage III breast cancer, without prior treatment, were enrolled in the study. Of the 70 patients enrolled tumor tissue from 45 individual patients was available for evaluation. The mean age of the patients was 49±9 years (between 29 and 64 years). The mean tumor size was 6.8±4.0 cm (between 2.3 and 21 cm). Patients were included in the study only if histopathologic assessment, performed as described in the Materials and Methods section, indicated adequate amounts of tumor tissue and homogenous pathology.

After enrollment, the patients were subjected to chemotherapy treatment with sequential doxorubicin 75 mg/m2 q2 wks×3 (+G-CSF days 2-11) and docetaxel 40 mg/m2 weekly×6 administration. The order of treatment was randomly assigned. 20 of 45 patients (44%) were first treated with doxorubicin followed by docetaxel treatment, while 25 of 45 patients (56%) were first treated with docetaxel following by doxorubicin treatment.

Materials and Methods

Fixed paraffin-embedded (FPE) tumor tissue from biopsy was obtained prior to and after chemotherapy. The pathologist selected the most representative primary tumor block, and submitted six 10 micron sections for RNA analysis. Specifically, a total of 6 sections (10 microns in thickness each) were prepared and placed in two Costar Brand Microcentrifuge Tubes (Polypropylene, 1.7 mL tubes, clear; 3 sections in each tube). If the tumor constituted less than 30% of the total specimen area, the sample may have been crudely dissected by the pathologist, using gross microdissection, putting the tumor tissue directly into the Costar tube.

mRNA was extracted and quantified by the RiboGreen® fluorescence method (Molecular probes). Molecular assays of quantitative gene expression were performed by RT-PCR, using the ABI PRISM 7900™ Sequence Detection System™ (Perkin-Elmer-Applied Biosystems, Foster City, Calif., USA). ABI PRISM 7900™ consists of a thermocycler, laser, charge-coupled device (CCD), camera and computer. The system amplifies samples in a 384-well format on a thermocycler. During amplification, laser-induced fluorescent signal is collected in real-time through fiber optics cables for all 384 wells, and detected at the CCD. The system includes software for running the instrument and for analyzing the data.

Analysis and Results

Tumor tissue was analyzed for 187 cancer-related genes and 5 reference genes. The threshold cycle (CT) values for each patient were normalized based on the median of the 5 reference genes for that particular patient. Patient beneficial response to chemotherapy was assessed by two different binary methods, by pathologic complete response, and by clinical complete response. Patients were formally assessed for response after week 6 and week 12 (at the completion of all chemotherapy.

A clinical complete response (cCR) requires complete disappearance of all clinically detectable disease, either by physical examination or diagnostic breast imaging.

A pathologic complete response (pCR) requires absence of residual breast cancer on histologic examination of biopsied breast tissue, lumpectomy or mastectomy specimens following primary chemotherapy. Residual DCIS may be present. Residual cancer in regional nodes may not be present.

A partial clinical response was defined as a ≧50% decrease in tumor area (sum of the products of the longest perpendicular diameters) or a ≧50% decrease in the sum of the products of the longest perpendicular diameters of multiple lesions in the breast and axilla. No area of disease may increase by >25% and no new lesions may appear.

When the pathological and clinical response data were in conflict with respect to the direction of predictive impact of a gene (i.e., negative versus positive) the pathologic response data were used, as pathologic response is a more rigorous measure of response to chemotherapy.

Pathologic response categories were:
0 Presence of detectable tumor following surgical resection {No CR}
1 Absence of detectable tumor following surgical resection {CR}

Complete clinical response categories were:
0 Presence of mass at end of treatment {No CR}
1 Absence of mass at end of treatment {CR}

Analysis was performed by: Analysis of the relationship between normalized gene expression and the binary outcomes of 0 or 1. Quantitative gene expression data were subjected to univariate analysis (t-test).

Table 1 presents pathologic response correlations with gene expression, and lists the 40 genes for which the p-value for the differences between the groups was <0.111. The first column of mean normalized expression {C_{T}} values pertains to patients who did not have a pathologic complete response The second column of mean normalized expression values pertains to patients who did have a pathologic complete response. The headings “p”, and “N” signify statistical p-value, and number of patients, respectively.

TABLE 1
Gene Expression and Pathologic Response

| | Mean | Mean | | N | N | Std. Dev. | Std. Dev. |
|---|---|---|---|---|---|---|---|
| | No CR | CR | p | No CR | CR | No CR | CR |
| VEGFC | −5.2 | −6.5 | 0.001 | 39 | 6 | 0.8 | 0.4 |
| B-Catenin | −1.6 | −2.3 | 0.013 | 39 | 6 | 0.6 | 0.6 |
| MMP2 | 0.2 | −1.0 | 0.016 | 39 | 6 | 1.1 | 1.3 |
| MMP9 | −3.4 | −1.5 | 0.016 | 39 | 6 | 1.5 | 3.2 |
| CNN | −4.4 | −5.7 | 0.023 | 39 | 6 | 1.3 | 1.0 |
| FLJ20354 | −5.7 | −4.7 | 0.024 | 39 | 6 | 1.0 | 1.0 |
| TGFB3 | −2.6 | −3.9 | 0.027 | 39 | 6 | 1.4 | 1.4 |
| PDGFRb | −2.2 | −3.2 | 0.029 | 39 | 6 | 1.0 | 1.2 |
| PLAUR | −3.9 | −4.6 | 0.033 | 39 | 6 | 0.7 | 0.6 |
| KRT19 | 1.7 | 0.3 | 0.033 | 39 | 6 | 1.4 | 1.6 |
| ID1 | −2.7 | −3.7 | 0.039 | 39 | 6 | 1.1 | 0.5 |
| RIZ1 | −3.8 | −4.6 | 0.039 | 39 | 6 | 0.8 | 1.2 |
| RAD54L | −5.9 | −5.0 | 0.039 | 39 | 6 | 0.9 | 1.0 |
| RB1 | −3.9 | −4.6 | 0.040 | 39 | 6 | 0.7 | 1.1 |
| SURV | −4.8 | −3.5 | 0.040 | 39 | 6 | 1.4 | 1.1 |
| EIF4EL3 | −3.6 | −4.0 | 0.042 | 39 | 6 | 0.4 | 0.4 |
| CYP2C8 | −7.2 | −6.6 | 0.044 | 39 | 6 | 0.4 | 1.8 |
| STK15 | −4.3 | −3.7 | 0.047 | 39 | 6 | 0.8 | 0.5 |
| ACTG2 | −4.6 | −6.1 | 0.049 | 39 | 6 | 1.8 | 0.9 |
| NEK2 | −5.2 | −4.2 | 0.060 | 39 | 6 | 1.2 | 1.0 |
| cMet | −6.5 | −7.3 | 0.061 | 39 | 6 | 0.9 | 0.2 |
| TIMP2 | 1.1 | 0.4 | 0.063 | 39 | 6 | 0.8 | 1.1 |
| C20 orf1 | −3.4 | −2.3 | 0.063 | 39 | 6 | 1.3 | 0.9 |
| DR5 | −5.3 | −5.9 | 0.066 | 39 | 6 | 0.7 | 0.6 |
| CD31 | −2.5 | −3.2 | 0.068 | 39 | 6 | 0.8 | 0.6 |
| BIN1 | −3.8 | −4.6 | 0.069 | 39 | 6 | 0.9 | 0.8 |
| COL1A2 | 2.4 | 1.3 | 0.073 | 39 | 6 | 1.3 | 1.4 |
| HIF1A | −2.9 | −3.4 | 0.074 | 39 | 6 | 0.6 | 0.4 |
| VIM | 0.7 | 0.2 | 0.079 | 39 | 6 | 0.7 | 0.9 |
| CDC20 | −3.7 | −2.5 | 0.080 | 39 | 6 | 1.6 | 0.8 |
| ID2 | −2.9 | −3.4 | 0.082 | 39 | 6 | 0.6 | 0.6 |
| MCM2 | −3.8 | −3.2 | 0.087 | 39 | 6 | 0.7 | 1.1 |
| CCNB1 | −4.5 | −3.8 | 0.088 | 39 | 6 | 0.9 | 0.6 |
| MYH11 | −3.8 | −5.0 | 0.094 | 39 | 6 | 1.8 | 1.3 |
| Chk2 | −5.0 | −4.6 | 0.095 | 39 | 6 | 0.6 | 0.8 |
| G-Catenin | −0.9 | −1.4 | 0.096 | 39 | 6 | 0.6 | 0.9 |
| HER2 | −0.7 | −1.8 | 0.100 | 39 | 6 | 1.4 | 1.6 |
| GSN | −2.1 | −2.8 | 0.109 | 39 | 6 | 1.0 | 1.0 |
| Ki-67 | −3.9 | −3.0 | 0.110 | 39 | 6 | 1.3 | 0.4 |
| TOP2A | −2.3 | −1.4 | 0.111 | 39 | 6 | 1.3 | 1.0 |

In the foregoing Table 1, genes exhibiting increased expression amongst CR pts, relative to NO CR pts are markers for increased likelihood of beneficial response to treatment, and genes exhibiting increased expression amongst NO CR pts, relative to CR pts are markers for decreased likelihood of beneficial response to treatment. For example, expression of VEGFC is higher in NO CR pt tumors relative to CR pt tumors {as indicated by a less negative normalized C_{T} value in the NO CR tumors}, and therefore increased expression of VEGFC gene {precisely, higher levels of VEGFC mRNA} predicts decreased likelihood of pt beneficial response to chemotherapy.

Based on the data set forth in Table 1, increased expression of the following genes correlates with increased likelihood of complete pathologic response to treatment: MMP9; FLJ20354; RAD54L; SURV; CYP2C8; STK15; NEK2; C20 orf1; CDC20; MCM2; CCNB1; Chk2; Ki-67; TOP2A, and increased expression of the following genes correlates with decreased likelihood of complete pathologic response to treatment: VEGFC; B-Catenin; MMP2; CNN; TGFB3; PDGFRb; PLAUR; KRT19; ID1; RIZ1; RB1; EIF4EL3; ACTG2; cMet; TIMP2; DR5; CD31; BIN1; COL1A2; HIF1A; VIM; ID2; MYH11; G-Catenin; HER2; GSN.

Table 2 presents the clinical response correlations with gene expression, and lists the genes for which the p-value for the differences between the groups was <0.095. The first column of mean normalized expression {CT} values pertains to patients who did not have a clinical complete response The second column of mean normalized expression values pertains to patients who did have a clinical complete response. The headings “p”, and “N” signify statistical p-value, and number of patients, respectively.

TABLE 2
Gene Expression and Clinical Response

| | | | | | | Std. | |
|---|---|---|---|---|---|---|---|
| | Mean | p | Valid | Valid N | Std. Dev. | Dev. | |
| Mean | No CR | CR | N | No CR | CR | No CR | CR |
| CCND1 | −1.2 | 0.5 | 0.000 | 25 | 20 | 1.3 | 1.3 |
| EstR1 | −3.8 | −0.9 | 0.000 | 25 | 20 | 2.9 | 1.9 |
| KRT18 | 0.5 | 1.7 | 0.000 | 25 | 20 | 1.2 | 0.9 |
| GATA3 | −2.2 | 0.2 | 0.001 | 25 | 20 | 2.4 | 1.6 |
| cIAP2 | −4.9 | −5.9 | 0.001 | 25 | 20 | 0.8 | 1.2 |
| KRT5 | −3.8 | −5.8 | 0.001 | 25 | 20 | 2.2 | 1.1 |
| RAB27B | −4.5 | −2.9 | 0.001 | 25 | 20 | 1.8 | 1.1 |
| IGF1R | −3.6 | −2.1 | 0.002 | 25 | 20 | 1.6 | 1.4 |
| cMet | −6.3 | −7.1 | 0.002 | 25 | 20 | 0.9 | 0.6 |
| HNF3A | −3.7 | −1.6 | 0.004 | 25 | 20 | 2.7 | 1.6 |
| CA9 | −5.4 | −6.9 | 0.004 | 25 | 20 | 2.1 | 1.1 |
| MCM3 | −5.6 | −6.2 | 0.005 | 25 | 20 | 0.8 | 0.6 |
| STMY3 | −1.7 | −0.2 | 0.006 | 25 | 20 | 1.9 | 1.5 |
| NPD009 | −4.5 | −3.3 | 0.006 | 25 | 20 | 1.6 | 1.2 |
| BAD | −3.2 | −2.8 | 0.008 | 25 | 20 | 0.6 | 0.4 |
| BBC3 | −5.3 | −4.7 | 0.009 | 25 | 20 | 0.8 | 0.7 |
| EGFR | −3.2 | −4.2 | 0.009 | 25 | 20 | 1.3 | 1.2 |
| CD9 | 0.2 | 0.7 | 0.010 | 25 | 20 | 0.6 | 0.6 |
| AKT1 | −1.2 | −0.7 | 0.013 | 25 | 20 | 0.7 | 0.6 |
| CD3z | −5.5 | −6.3 | 0.014 | 25 | 20 | 1.0 | 1.3 |
| KRT14 | −3.6 | −5.3 | 0.014 | 25 | 20 | 2.7 | 1.4 |
| DKFZp564 | −4.9 | −5.8 | 0.015 | 25 | 20 | 1.1 | 1.2 |
| Bcl2 | −3.6 | −2.6 | 0.016 | 25 | 20 | 1.3 | 1.4 |
| BECN1 | −2.4 | −2.0 | 0.017 | 25 | 20 | 0.7 | 0.5 |
| KLK10 | −5.0 | −6.5 | 0.017 | 25 | 20 | 2.5 | 1.2 |
| DIABLO | −4.7 | −4.3 | 0.019 | 25 | 20 | 0.6 | 0.6 |
| MVP | −2.5 | −1.9 | 0.021 | 25 | 20 | 0.7 | 0.8 |
| VEGFB | −2.5 | −1.9 | 0.021 | 25 | 20 | 0.9 | 0.5 |
| ErbB3 | −2.8 | −2.0 | 0.021 | 25 | 20 | 1.2 | 0.8 |
| MDM2 | −1.3 | −0.7 | 0.021 | 25 | 20 | 0.7 | 1.0 |
| Bclx | −2.7 | −2.3 | 0.022 | 25 | 20 | 0.6 | 0.7 |
| CDH | −3.0 | −2.1 | 0.022 | 25 | 20 | 1.0 | 1.4 |
| HLA-DPB1 | 0.9 | 0.3 | 0.022 | 25 | 20 | 0.9 | 0.9 |
| PR | −5.4 | −3.9 | 0.026 | 25 | 20 | 2.1 | 2.1 |
| KRT17 | −3.3 | −4.8 | 0.027 | 25 | 20 | 2.6 | 1.4 |
| GSTp | −0.8 | −1.5 | 0.029 | 25 | 20 | 0.8 | 1.1 |
| IRS1 | −3.7 | −2.8 | 0.034 | 25 | 20 | 1.4 | 1.4 |
| NFKBp65 | −2.4 | −2.1 | 0.039 | 25 | 20 | 0.6 | 0.4 |
| IGFBP2 | −1.9 | −0.9 | 0.040 | 25 | 20 | 1.7 | 1.3 |
| RPS6KB1 | −5.3 | −4.9 | 0.042 | 25 | 20 | 0.8 | 0.5 |
| BIN1 | −3.7 | −4.2 | 0.043 | 25 | 20 | 0.9 | 0.9 |
| CD31 | −2.4 | −2.9 | 0.046 | 25 | 20 | 0.8 | 0.9 |
| G-Catenin | −1.2 | −0.8 | 0.049 | 25 | 20 | 0.6 | 0.7 |
| DHPS | −2.6 | −2.2 | 0.054 | 25 | 20 | 0.8 | 0.5 |
| TIMP3 | 0.7 | 1.4 | 0.054 | 25 | 20 | 1.2 | 1.0 |
| ZNF217 | −1.1 | −0.6 | 0.058 | 25 | 20 | 0.8 | 0.8 |
| KIAA1209 | −4.2 | −4.8 | 0.061 | 25 | 20 | 1.0 | 1.0 |
| CYP2C8 | −7.3 | −6.9 | 0.061 | 25 | 20 | 0.3 | 1.1 |
| COX2 | −7.3 | −7.5 | 0.063 | 25 | 20 | 0.4 | 0.1 |
| RB1 | −4.2 | −3.8 | 0.063 | 25 | 20 | 1.0 | 0.5 |
| ACTG2 | −4.4 | −5.3 | 0.065 | 25 | 20 | 2.0 | 1.2 |
| pS2 | −3.9 | −1.9 | 0.068 | 25 | 20 | 3.6 | 3.2 |
| COL1A2 | 1.9 | 2.7 | 0.069 | 25 | 20 | 1.4 | 1.3 |
| BRK | −5.5 | −4.9 | 0.070 | 25 | 20 | 1.0 | 1.2 |
| CEGP1 | −4.8 | −3.5 | 0.073 | 25 | 20 | 2.5 | 2.4 |
| EPHX1 | −2.0 | −1.6 | 0.078 | 25 | 20 | 0.8 | 0.8 |
| VEGF | −0.3 | −0.8 | 0.084 | 25 | 20 | 0.9 | 0.8 |
| TP53BP1 | −3.3 | −2.9 | 0.085 | 25 | 20 | 0.8 | 0.7 |
| COL1A1 | 4.3 | 5.0 | 0.089 | 25 | 20 | 1.4 | 1.1 |
| FGFR1 | −3.6 | −2.8 | 0.090 | 25 | 20 | 1.2 | 1.8 |
| CTSL2 | −5.6 | −6.4 | 0.095 | 25 | 20 | 1.7 | 1.0 |

Based on the data set forth in Table 2, increased expression of the following genes correlates with increased likelihood of complete clinical response to treatment: CCND1; EstR1; KRT18; GATA3; RAB27B; IGF1R; HNF3A; STMY3; NPD009; BAD; BBC3; CD9; AKT1; Bcl2; BECN1; DIABLO; MVP; VEGFB; ErbB3; MDM2; Bclx; CDH1; PR; IRS1; NFKBp65; IGFBP2; RPS6 KB1; DHPS; TIMP3; ZNF217; CYP2C8; pS2; BRK; CEGP1; EPHX1; TP53BP1; COL1A1; and FGFR1
and increased expression of the following genes correlates with decreased likelihood of complete clinical response to treatment: cIAP2; KRT5; CA9; MCM3; EGFR; CD3z; KRT14; DKFZp564; KLK10; HLA-DPB1; KRT17; GSTp; BIN1; CD31; KIAA1209; COX2; VEGF; and CTSL2.

All references cited throughout the disclosure are hereby expressly incorporated by reference.

While the invention has been described with emphasis upon certain specific embodiments, it is be apparent to those skilled in the art that variations and modification in the specific methods and techniques are possible. Accordingly, this invention includes all modifications encompassed within the spirit and scope of the invention as defined by the following claims.

TABLE 3

| | | | | | |
|---|---|---|---|---|---|
| Name | Accession | Name | SEQ ID Nos. | Sequence | Length |

| | | | | | |
|---|---|---|---|---|---|
| ACTG2 | NM_001615 | S4543/ACTG2.f3 | SEQ ID NO: 1 | ATGTACGTCGCCATTCAAGCT | 21 |
| ACTG2 | NM_001615 | S4544/ACTG2.r3 | SEQ ID NO: 2 | ACGCCATCACCTGAATCCA | 19 |
| ACTG2 | NM_001615 | S4545/ACTG2.p3 | SEQ ID NO: 3 | CTGGCCGCACGACAGGCATC | 20 |
| AKT1 | NM_005163 | S0010/AKT1.f3 | SEQ ID NO: 4 | CGCTTCTATGGCGCTGAGAT | 20 |
| AXT1 | NM_005163 | S0012/AKT1.r3 | SEQ ID NO: 5 | TCCCGGTACACCACGTTCTT | 20 |
| AKT1 | NM_005163 | S4776/AKT1.p3 | SEQ ID NO: 6 | CAGCCCTGGACTACCTGCACTCGG | 24 |
| B-Catenin | NM_001904 | S2150/B-Cate.f3 | SEQ ID NO: 7 | GGCTCTTGTGCGTACTGTCCTT | 22 |
| B-Catenin | NM_001904 | S2151/B-Cate.r3 | SEQ ID NO: 8 | TCAGATGACGAAGAGCACAGATG | 23 |
| B-Catenin | NM_001904 | S5046/B-Cate.p3 | SEQ ID NO: 9 | AGGCTCAGTGATGTCTTCCCTGTCACCAG | 29 |
| BAD | NM_032989 | S2011/BAD.f1 | SEQ ID NO: 10 | GGGTCAGGTGCCTCGAGAT | 19 |
| BAD | NM_032989 | S2012/BAD.r1 | SEQ ID NO: 11 | CTGCTCACTCGGCTCAAACTC | 21 |
| BAD | NM_032989 | S5058/BAD.p1 | SEQ ID NO: 12 | TGGGCCCAGAGCATGTTCCAGATC | 24 |
| BBC3 | NM_014417 | S1584/BBC3.f2 | SEQ ID NO: 13 | CCTGGAGGGTCCTGTACAAT | 20 |
| BBC3 | NM_014417 | S1585/BBC3.r2 | SEQ ID NO: 14 | CTAATTGGGCTCCATCTCG | 19 |
| BBC3 | NM_014417 | S4890/BBC3.p2 | SEQ ID NO: 15 | CATCATGGGACTCCTGCCCTTACC | 24 |
| Bcl2 | NM_000633 | S0043/Bcl2.f2 | SEQ ID NO: 16 | CAGATGGACCTAGTACCCACTGAGA | 25 |
| Bcl2 | NM_000633 | S0045/Bcl2.r2 | SEQ ID NO: 17 | CCTATGATTTAAGGGCATTTTTCC | 24 |
| Bcl2 | NM_000633 | S4732/Bcl2.p2 | SEQ ID NO: 18 | TTCCACGCCGAAGGACAGCGAT | 22 |
| Bclx | NM_001191 | S0046/Bclx.f2 | SEQ ID NO: 19 | CTTTTGTGGAACTCTATGGGAACA | 24 |
| Bclx | NM_001191 | S0048/Bclx.r2 | SEQ ID NO: 20 | CAGCGGTTGAAGCGTTCCT | 19 |
| Bclx | NM_001191 | S4898/Bclx.p2 | SEQ ID NO: 21 | TTCGGCTCTCGGCTGCTGCA | 20 |
| BECN1 | NM_003766 | S2642/BECN1.f3 | SEQ ID NO: 22 | CAGTTTGGCACAATCAATAACTTCA | 25 |
| BECN1 | NM_003766 | S2643/BECN1.r3 | SEQ ID NO: 23 | GCAGCATTAATCTCATTCCATTCC | 24 |
| BECN1 | NM_003766 | S4953/BECN1.p3 | SEQ ID NO: 24 | TCGCCTGCCCAGTGTTCCCG | 20 |
| BIN1 | NM_004305 | S2651/BIN1.f3 | SEQ ID NO: 25 | CCTGCAAAAGGGAACAAGAG | 20 |
| BIN1 | NM_004305 | S2652/BIN1.r3 | SEQ ID NO: 26 | CGTGGTTGACTCTGATCTCG | 20 |
| BIN1 | NM_004305 | S4954/BIN1.p3 | SEQ ID NO: 27 | CTTCGCCTCCAGATGGCTCCC | 21 |
| BRK | NM_005975 | S0678/BRK.f2 | SEQ ID NO: 28 | GTGCAGGAAAGGTTCACAAA | 20 |
| BRK | NM_005975 | S0679/BRK.r2 | SEQ ID NO: 29 | GCACACACGATGGAGTAAGG | 20 |
| BRK | NM_005975 | S4789/BRK.p2 | SEQ ID NO: 30 | AGTGTCTGCGTCCAATACACGCGT | 24 |
| C20 orf1 | NM_012112 | S3560/C20 or.f1 | SEQ ID NO: 31 | TCAGCTGTGAGCTGCGGATA | 20 |
| C20 orf1 | NM_012112 | S3561/C20 or.r1 | SEQ ID NO: 32 | ACGGTCCTAGGTTTGAGGTTAAGA | 24 |
| C20 orf1 | NM_012112 | S3562/C20 or.p1 | SEQ ID NO: 33 | CAGGTCCCATTGCCGGGCG | 19 |
| CA9 | NM_001216 | S1398/CA9.f3 | SEQ ID NO: 34 | ATCCTAGCCCTGGTTTTTGG | 20 |
| CA9 | NM_001216 | S1399/CA9.r3 | SEQ ID NO: 35 | CTGCCTTCTCATCTGCACAA | 20 |
| CA9 | NM_001216 | S4938/CA9.p3 | SEQ ID NO: 36 | TTTGCTGTCACCAGCGTCGC | 20 |
| CCNB1 | NM_031966 | S1720/CCNB1.f2 | SEQ ID NO: 37 | TTCAGGTTGTTGCAGGAGAC | 20 |
| CCNB1 | NM_031966 | S1721/CCNB1.r2 | SEQ ID NO: 38 | CATCTTCTTGGGCACACAAT | 20 |
| CCNB1 | NM_031966 | S4733/CCNB1.p2 | SEQ ID NO: 39 | TGTCTCCATTATTGATCGGTTCATGCA | 27 |
| CCND1 | NM_001758 | S0058/CCND1.f3 | SEQ ID NO: 40 | GCATGTTCGTGGCCTCTAAGA | 21 |
| COND1 | NM_001758 | S0060/CCND1.r3 | SEQ ID NO: 41 | CGGTGTAGATGCACAGCTTCTC | 22 |
| COND1 | NM_001758 | S4986/CCND1.p3 | SEQ ID NO: 42 | AAGGAGACCATCCCCCTGACGGC | 23 |
| CD31 | NM_000442 | S1407/CD31.f3 | SEQ ID NO: 43 | TGTATTTCAAGACCTCTGTGCACTT | 25 |
| CD31 | NM_000442 | S1408/CD31.r3 | SEQ ID NO: 44 | TTAGCCTGAGGAATTGCTGTGTT | 23 |
| CD31 | NM_000442 | S4939/CD31.p3 | SEQ ID NO: 45 | TTTATGAACCTGCCCTGCTCCCACA | 25 |
| CD3z | NM_000734 | S0064/CD3z.f1 | SEQ ID NO: 46 | AGATGAAGTGGAAGGCGCTT | 20 |
| CD3z | NM_000734 | S0066/CD3z.r1 | SEQ ID NO: 47 | TGCCTCTGTAATCGGCAACTG | 21 |
| CD3z | NM_000734 | S4988/CD3z.p1 | SEQ ID NO: 48 | CACCGCGGCCATCCTGCA | 18 |
| CD9 | NM_001769 | S0686/CD9.f1 | SEQ ID NO: 49 | GGGCGTGGAACAGTTTATCT | 20 |
| CD9 | NM_001769 | S0687/CD9.r1 | SEQ ID NO: 50 | CACGGTGAAGGTTTCGAGT | 19 |
| CD9 | NM_001769 | S4792/CD9.p1 | SEQ ID NO: 51 | AGACATCTGCCCCAAGAAGGACGT | 24 |
| CDC20 | NM_001255 | S4447/CDC20.f1 | SEQ ID NO: 52 | TGGATTGGAGTTCTGGGAATG | 21 |
| CDC20 | NM_001255 | S4448/CDC20.r1 | SEQ ID NO: 53 | GCTTGCACTCCACAGGTACACA | 22 |
| CDC20 | NM_001255 | S4449/CDC20.p1 | SEQ ID NO: 54 | ACTGGCCGTGGCACTGGACAACA | 23 |
| CDH1 | NM_004360 | S0073/CDH1.f3 | SEQ ID NO: 55 | TGAGTGTCCCCCGGTATCTTC | 21 |
| CDH1 | NM_004360 | S0075/CDH1.r3 | SEQ ID NO: 56 | CAGCCGCTTTCAGATTTTCAT | 21 |
| CDH1 | NM_004360 | S4990/CDH1.p3 | SEQ ID NO: 57 | TGCCAATCCCGATGAAATTGGAAATTT | 27 |
| CEGP1 | NM_020974 | S1494/CEGP1.f2 | SEQ ID NO: 58 | TGACAATCAGCACACCTGCAT | 21 |
| CEGP1 | NM_020974 | S1495/CEGP1.r2 | SEQ ID NO: 59 | TGTGACTACAGCCGTGATCCTTA | 23 |
| CEGP1 | NM_020974 | S4735/CEGP1.p2 | SEQ ID NO: 60 | CAGGCCCTCTTCCGAGCGGT | 20 |
| Chk2 | NM_007194 | S1434/Chk2.f3 | SEQ ID NO: 61 | ATGTGGAACCCCCACCTACTT | 21 |
| Chk2 | NM_007194 | S1435/Chk2.r3 | SEQ ID NO: 62 | CAGTCCACAGCACGGTTATACC | 22 |
| Chk2 | NM_007194 | S4942/Chk2.p3 | SEQ ID NO: 63 | AGTCCCAACAGAAACAAGAACTTCAGGCG | 29 |
| cIAP2 | NM_001165 | S0076/cIAP2.f2 | SEQ ID NO: 64 | GGATATTTCCGTGGCTCTTATTCA | 24 |
| cIAP2 | NM_001165 | S0078/cIAP2.r2 | SEQ ID NO: 65 | CTTCTCATCAAGGCAGAAAAATCTT | 25 |
| cIAP2 | NM_001165 | S4991/cIAP2.p2 | SEQ ID NO: 66 | TCTCCATCAAATCCTGTAAACTCCAGAGCA | 30 |
| cMet | NM_000245 | S0082/cMet.f2 | SEQ ID NO: 67 | GACATTTCCAGTCCTGCAGTCA | 22 |
| cMet | NM_000245 | S0084/cMet.r2 | SEQ ID NO: 68 | CTCCGATCGCACACATTTGT | 20 |
| cMet | NM_000245 | S4993/cMet.p2 | SEQ ID NO: 69 | TGCCTCTCTGCCCCACCCTTTGT | 23 |
| CNN | NM_001299 | S4564/CNN.f1 | SEQ ID NO: 70 | TCCACCCTCCTGGCTTTG | 18 |
| CNN | NM_001299 | S4565/CNN.r1 | SEQ ID NO: 71 | TCACTCCCACGTTCACCTTGT | 21 |
| CNN | NM_001299 | S4566/CNN.p1 | SEQ ID NO: 72 | TCCTTTCGTCTTCGCCATGCTGG | 23 |
| COL1A1 | NM_000088 | S4531/COL1A1.f1 | SEQ ID NO: 73 | GTGGCCATCCAGCTGACC | 18 |
| COL1A1 | NM_000088 | S4532/COL1A1.r1 | SEQ ID NO: 74 | CAGTGGTAGGTGATGTTCTGGGA | 23 |
| COL1A1 | NM_000088 | S4533/COL1A1.p1 | SEQ ID NO: 75 | TCCTGCGCCTGATGTCCACCG | 21 |
| COL1A2 | NM_000089 | S4534/COL1A2.f1 | SEQ ID NO: 76 | CAGCCAAGAACTGGTATAGGAGCT | 24 |
| COL1A2 | NM_000089 | S4535/COL1A2.r1 | SEQ ID NO: 77 | AAACTGGCTGCCAGCATTG | 19 |
| COL1A2 | NM_000089 | S4536/COL1A2.p1 | SEQ ID NO: 78 | TCTCCTAGCCAGACGTGTTTCTTGTCCTTG | 30 |
| COX2 | NM_000963 | S0088/COX2.f1 | SEQ ID NO: 79 | TCTGCAGAGTTGGAAGCACTCTA | 23 |
| COX2 | NM_000963 | S0090/COX2.r1 | SEQ ID NO: 80 | GCCGAGGCTTTTCTACCAGAA | 21 |
| COX2 | NM_000963 | S4995/COX2.p1 | SEQ ID NO: 81 | CAGGATACAGCTCCACAGCATCGATGTC | 28 |
| CTSL2 | NM_001333 | S4354/CTSL2.f1 | SEQ ID NO: 82 | TGTCTCACTGAGCGAGCAGAA | 21 |
| CTSL2 | NM_001333 | S4355/CTSL2.r1 | SEQ ID NO: 83 | ACCATTGCAGCCCTGATTG | 19 |
| CTSL2 | NM_001333 | S4356/CTSL2.p1 | SEQ ID NO: 84 | CTTGAGGACGCGAACAGTCCACCA | 24 |
| CYP2C8 | NM_000770 | S1470/CYP2C8.f2 | SEQ ID NO: 85 | CCGTGTTCAAGAGGAAGCTC | 20 |
| CYP2C8 | NM_000770 | S1471/CYP2C8.r2 | SEQ ID NO: 86 | AGTGGGATCACAGGGTGAAG | 20 |
| CYP2C8 | NM_000770 | S4946/CYP2C8.p2 | SEQ ID NO: 87 | TTTTCTCAACTCCTCCACAAGGCA | 24 |
| DHPS | NM_013407 | S4519/DHPS.f3 | SEQ ID NO: 88 | GGGAGAACGGGATCAATAGGAT | 22 |
| DHPS | NM_013407 | S4520/DHPS.r3 | SEQ ID NO: 89 | GCATCAGCCAGTCCTCAAACT | 21 |
| DHPS | NM_013407 | S4521/DHPS.p3 | SEQ ID NO: 90 | CTCATTGGGCACCAGCAGGTTTCC | 24 |
| DIABLO | NM_019887 | S0808/DIABLO.f1 | SEQ ID NO: 91 | CACAATGGCGGCTCTGAAG | 19 |
| DIABLO | NM_019887 | S0809/DIABLO.r1 | SEQ ID NO: 92 | ACACAAACACTGTCTGTACCTGAAGA | 26 |
| DIABLO | NM_019887 | S4813/DIABLO.p1 | SEQ ID NO: 93 | AAGTTACGCTGCGCGACAGCCAA | 23 |
| DKFZp564 | XM_047080 | S4405/DKFZp5.f2 | SEQ ID NO: 94 | CAGTGCTTCCATGGACAAGT | 20 |
| DKFZp564 | XM_047080 | S4406/DKFZp5.r2 | SEQ ID NO: 95 | TGGACAGGGATGATTGATGT | 20 |
| DKFZp564 | XM_047080 | S4407/DKFZp5.p2 | SEQ ID NO: 96 | ATCTCCATCAGCATGGGCCAGTTT | 24 |
| DR5 | NM_003842 | S2551/DR5.f2 | SEQ ID NO: 97 | CTCTGAGACAGTGCTTCGATGACT | 24 |
| DR5 | NM_003842 | S2552/DR5.r2 | SEQ ID NO: 98 | CCATGAGGCCCAACTTCCT | 19 |
| DR5 | NM_003842 | S4979/DR5.p2 | SEQ ID NO: 99 | CAGACTTGGTGCCCTTTGACTCC | 23 |
| EGFR | NM_005228 | S0103/EGFR.f2 | SEQ ID NO: 100 | TGTCGATGGACTTCCAGAAC | 20 |
| EGFR | NM_005228 | S0105/EGFR.r2 | SEQ ID NO: 101 | ATTGGGACAGCTTGGATCA | 19 |
| EGFR | NM_005228 | S4999/EGFR.p2 | SEQ ID NO: 102 | CACCTGGGCAGCTGCCAA | 18 |
| EIF4EL3 | NM_004846 | S4495/EIF4EL.f1 | SEQ ID NO: 103 | AAGCCGCGGTTGAATGTG | 18 |
| EIF4EL3 | NM_004846 | S4496/EIF4EL.r1 | SEQ ID NO: 104 | TGACGCCAGCTTCAATGATG | 20 |
| EIF4EL3 | NM_004846 | S4497/EIF4EL.p1 | SEQ ID NO: 105 | TGACCCTCTCCCTCTCTGGATGGCA | 25 |
| EPHX1 | NM_000120 | S1865/EPHX1.f2 | SEQ ID NO: 106 | ACCGTAGGCTCTGCTCTGAA | 20 |
| EPHX1 | NM_000120 | S1866/EPHX1.r2 | SEQ ID NO: 107 | TGGTCCAGGTGGAAAACTTC | 20 |
| EPHX1 | NM_000120 | S4754/EPHX1.p2 | SEQ ID NO: 108 | AGGCAGCCAGACCCACAGGA | 20 |
| ErbB3 | NM_001982 | S0112/ErbB3.f1 | SEQ ID NO: 109 | CGGTTATGTCATGCCAGATACAC | 23 |
| ErbB3 | NM_001982 | S0114/ErbB3.r1 | SEQ ID NO: 110 | GAACTGAGACCCACTGAAGAAAGG | 24 |
| ErbB3 | NM_001982 | S5002/ErbB3.p1 | SEQ ID NO: 111 | CCTCAAAGGTACTCCCTCCTCCCGG | 25 |
| EstR1 | NM_000125 | S0115/EstR1.f1 | SEQ ID NO: 112 | CGTGGTGCCCCTCTATGAC | 19 |
| EstR1 | NM_000125 | S0117/EstR1.r1 | SEQ ID NO: 113 | GGCTAGTGGGCGCATGTAG | 19 |
| EstR1 | NM_000125 | S4737/EstR1.p1 | SEQ ID NO: 114 | CTGGAGATGCTGGACGCCC | 19 |
| FGFR1 | NM_023109 | S0818/FGFR1.f3 | SEQ ID NO: 115 | CACGGGACATTCACCACATC | 20 |
| FGFR1 | NM_023109 | S0819/FGFR1.r3 | SEQ ID NO: 116 | GGGTGCCATCCACTTCACA | 19 |
| FGFR1 | NM_023109 | S4816/FGFR1.p3 | SEQ ID NO: 117 | ATAAAAAGACAACCAACGGCCGACTGC | 27 |
| FLJ20354 | NM_017779 | S4309/FLJ203.f1 | SEQ ID NO: 118 | GCGTATGATTTCCCGAATGAG | 21 |
| FLJ20354 | NM_017779 | S4310/FLJ203.r1 | SEQ ID NO: 119 | CAGTGACCTCGTACCCATTGC | 21 |
| FLJ20354 | NM_017779 | S4311/FLJ203.p1 | SEQ ID NO: 120 | ATGTTGATATGCCCAAACTTCATGA | 25 |
| G-Catenin | NM_002230 | S2153/G-Cate.f1 | SEQ ID NO: 121 | TCAGCAGCAAGGGCATCAT | 19 |
| G-Catenin | NM_002230 | S2154/G-Cate.r1 | SEQ ID NO: 122 | GGTGGTTTTCTTGAGCGTGTACT | 23 |
| G-Catenin | NM_002230 | S5044/G-Cate.p1 | SEQ ID NO: 123 | CGCCCGCAGGCCTCATCCT | 19 |
| GATA3 | NM_002051 | S0127/GATA3.f3 | SEQ ID NO: 124 | CAAAGGAGCTCACTGTGGTGTCT | 23 |
| GATA3 | NM_002051 | S0129/GATA3.r3 | SEQ ID NO: 125 | GAGTCAGAATGGCTTATTCACAGATG | 26 |
| GATA3 | NM_002051 | S5005/GATA3.p3 | SEQ ID NO: 126 | TGTTCCAACCACTGAATCTGGACC | 24 |
| GSN | NM_000177 | S2679/GSN.f3 | SEQ ID NO: 127 | CTTCTGCTAAGCGGTACATCGA | 22 |
| GSN | NM_000177 | S2680/GSN.r3 | SEQ ID NO: 128 | GGCTCAAAGCCTTGCTTCAC | 20 |
| GSN | NM_000177 | S4957/GSN.p3 | SEQ ID NO: 129 | ACCCAGCCAATCGGGATCGGC | 21 |
| GSTp | NM_000852 | S0136/GSTp.f3 | SEQ ID NO: 130 | GAGACCCTGCTGTCCCAGAA | 20 |
| GSTp | NM_000852 | S0138/GSTp.r3 | SEQ ID NO: 131 | GGTTGTAGTCAGCGAAGGAGATC | 23 |
| GSTp | NM_000852 | S5007/GSTp.p3 | SEQ ID NO: 132 | TCCCACAATGAAGGTCTTGCCTCCCT | 26 |
| HER2 | NM_004448 | S0142/HER2.f3 | SEQ ID NO: 133 | CGGTGTGAGAAGTGCAGCAA | 20 |
| HER2 | NM_004448 | S0144/HER2.r3 | SEQ ID NO: 134 | CCTCTCGCAAGTGCTCCAT | 19 |
| HER2 | NM_004448 | S4729/HER2.p3 | SEQ ID NO: 135 | CCAGACCATAGCACACTCGGGCAC | 24 |
| HIF1A | NM_001530 | S1207/HIF1A.f3 | SEQ ID NO: 136 | TGAACATAAAGTCTGCAACATGGA | 24 |
| HIF1A | NM_001530 | S1208/HIF1A.r3 | SEQ ID NO: 137 | TGAGGTTGGTTACTGTTGGTATCATATA | 28 |
| HIF1A | NM_001530 | S4753/H1F1A.p3 | SEQ ID NO: 138 | TTGCACTGCACAGGCCACATTCAC | 24 |
| HLA-DPB1 | NM_002121 | S4573/HLA-DP.f1 | SEQ ID NO: 139 | TCCATGATGGTTCTGCAGGTT | 21 |
| HLA-DPB1 | NM_002121 | S4574/HLA-DP.r1 | SEQ ID NO: 140 | TGAGCAGCACCATCAGTAACG | 21 |
| HLA-DPB1 | NM_002121 | S4575/HLA-DP.p1 | SEQ ID NO: 141 | CCCCGGACAGTGGCTCTGACG | 21 |
| HNF3A | NM_004496 | S0148/HNF3A.f1 | SEQ ID NO: 142 | TCCAGGATGTTAGGAACTGTGAAG | 24 |
| HNF3A | NM_004496 | S0150/HNF3A.r1 | SEQ ID NO: 143 | GCGTGTCTGCGTAGTAGCTGTT | 22 |
| HNF3A | NM_004496 | S5008/HNF3A.p1 | SEQ ID NO: 144 | AGTCGCTGGTTTCATGCCCTTCCA | 24 |
| ID1 | NM_002165 | S0820/ID1.f1 | SEQ ID NO: 145 | AGAACCGCAAGGTGAGCAA | 19 |
| ID1 | NM_002165 | S0821/ID1.r1 | SEQ ID NO: 146 | TCCAACTGAAGGTCCCTGATG | 21 |
| ID1 | NM_002165 | S4832/ID1.p1 | SEQ ID NO: 147 | TGGAGATTCTCCAGCACGTCATCGAC | 26 |
| ID2 | NM_002166 | S0151/ID2.f4 | SEQ ID NO: 148 | AACGACTGCTACTCCAAGCTCAA | 23 |
| ID2 | NM_002166 | S0153/ID2.r4 | SEQ ID NO: 149 | GGATTTCCATCTTGCTCACCTT | 22 |
| ID2 | NM_002166 | S5009/ID2.p4 | SEQ ID NO: 150 | TGCCCAGCATCCCCCAGAACAA | 22 |
| IGF1R | NM_000875 | S1249/IGF1R.f3 | SEQ ID NO: 151 | GCATGGTAGCCGAAGATTTCA | 21 |
| IGF1R | NM_000875 | S1250/IGF1R.r3 | SEQ ID NO: 152 | TTTCCGGTAATAGTCTGTCTCATAGATATC | 30 |
| IGF1R | NM_000875 | S4895/IGF1R.p3 | SEQ ID NO: 153 | CGCGTCATACCAAAATCTCCGATTTTGA | 28 |
| IGFBP2 | NM_000597 | S1128/IGFBP2.f1 | SEQ ID NO: 154 | GTGGACAGCACCATGAACA | 19 |
| IGFBP2 | NM_000597 | S1129/IGFBP2.r1 | SEQ ID NO: 155 | CCTTCATACCCGACTTGAGG | 20 |
| IGFBP2 | NM_000597 | S4837/IGFBP2.p1 | SEQ ID NO: 156 | CTTCCGGCCAGCACTGCCTC | 20 |
| IRS1 | NM_005544 | S1943/IRS1.f3 | SEQ ID NO: 157 | CCACAGCTCACCTTCTGTCA | 20 |
| IRS1 | NM_005544 | S1944/IRS1.r3 | SEQ ID NO: 158 | CCTCAGTGCCAGTCTCTTCC | 20 |
| IRS1 | NM_005544 | S5050/IRS1.p3 | SEQ ID NO: 159 | TCCATCCCAGCTCCAGCCAG | 20 |
| Ki-67 | NM_002417 | S0436/Ki-67.f2 | SEQ ID NO: 160 | CGGACTTTGGGTGCGACTT | 19 |
| Ki-67 | NM_002417 | S0437/Ki-67.r2 | SEQ ID NO: 161 | TTACAACTCTTCCACTGGGACGAT | 24 |
| Ki-67 | NM_002417 | S4741/Ki-67.p2 | SEQ ID NO: 162 | CCACTTGTCGAACCACCGCTCGT | 23 |
| KIAA1209 | AJ420468 | S4438/KIAA12.f1 | SEQ ID NO: 163 | GCCTAGCAGTTCTACCATGATCAG | 24 |
| KIAA1209 | AJ420468 | S4439/KIAA12.r1 | SEQ ID NO: 164 | GGTGATCGGTCCAGATGTTTCT | 22 |
| KIAA1209 | AJ420468 | S4440/K1AA12.p1 | SEQ ID NO: 165 | AGAGCTCCACCCGCTCGAAGCA | 22 |
| KLK10 | NM_002776 | S2624/KLK10.f3 | SEQ ID NO: 166 | GCCCAGAGGCTCCATCGT | 18 |
| KLK10 | NM_002776 | S2625/KLK10.r3 | SEQ ID NO: 167 | CAGAGGTTTGAACAGTGCAGACA | 23 |
| KLK10 | NM_002776 | S4978/KLK10.p3 | SEQ ID NO: 168 | CCTCTTCCTCCCCAGTCGGCTGA | 23 |
| KRT14 | NM_000526 | S1853/KRT14.f1 | SEQ ID NO: 169 | GGCCTGCTGAGATCAAAGAC | 20 |
| KRT14 | NM_000526 | S1854/KRT14.r1 | SEQ ID NO: 170 | GTCCACTGTGGCTGTGAGAA | 20 |
| KRT14 | NM_000526 | S5037/KRT14.p1 | SEQ ID NO: 171 | TGTTCCTCAGGTCCTCAATGGTCTTG | 26 |
| KRT17 | NM_000422 | S0172/KRT17.f2 | SEQ ID NO: 172 | CGAGGATTGGTTCTTCAGCAA | 21 |
| KRT17 | NM_000422 | S0174/KRT17.r2 | SEQ ID NO: 173 | ACTCTGCACCAGCTCACTGTTG | 22 |
| KRT17 | NM_000422 | S5013/KRT17.p2 | SEQ ID NO: 174 | CACCTCGCGGTTCAGTTCCTCTGT | 24 |
| KRT18 | NM_000224 | S1710/KRT18.f2 | SEQ ID NO: 175 | AGAGATCGAGGCTCTCAAGG | 20 |
| KRT18 | NM_000224 | S1711/KRT18.r2 | SEQ ID NO: 176 | GGCCTTTTACTTCCTCTTCG | 20 |
| KRT18 | NM_000224 | S4762/KRT18.p2 | SEQ ID NO: 177 | TGGTTCTTCTTCATGAAGAGCAGCTCC | 27 |
| KRT19 | NM_002276 | S1515/KRT19.f3 | SEQ ID NO: 178 | TGAGCGGCAGAATCAGGAGTA | 21 |
| KRT19 | NM_002276 | S1516/KRT19.r3 | SEQ ID NO: 179 | TGCGGTAGGTGGCAATCTC | 19 |
| KRT19 | NM_002276 | S4866/KRT19.p3 | SEQ ID NO: 180 | CTCATGGACATCAAGTCGCGGCTG | 24 |
| KRT5 | NM_000424 | S0175/KRT5.f3 | SEQ ID NO: 181 | tcagtggagaaggagttgga | 20 |
| KRT5 | NM_000424 | S0177/KRT5.r3 | SEQ ID NO: 182 | tgccatatccagaggaaaca | 20 |
| KRT5 | NM_000424 | S5015/KRT5.p3 | SEQ ID NO: 183 | ccagtcaacatctctgttgtcacaagca | 28 |
| MCM2 | NM_004526 | S1602/MCM2.f2 | SEQ ID NO: 184 | GACTTTTGCCCGCTACCTTTC | 21 |
| MCM2 | NM_004526 | S1603/MCM2.r2 | SEQ ID NO: 185 | GCCACTAACTGCTTCAGTATGAAGAG | 26 |
| MCM2 | NM_004526 | S4900/MCM2.p2 | SEQ ID NO: 186 | ACAGCTCATTGTTGTCACGCCGGA | 24 |
| MCM3 | NM_002388 | S1524/MCM3.f3 | SEQ ID NO: 187 | GGAGAACAATCCCCTTGAGA | 20 |
| MCM3 | NM_002388 | S1525/MCM3.r3 | SEQ ID NO: 188 | ATCTCCTGGATGGTGATGGT | 20 |
| MCM3 | NM_002388 | S4870/MCM3.p3 | SEQ ID NO: 189 | TGGCCTTTCTGTCTACAAGGATCACCA | 27 |
| MDM2 | NM_002392 | S0830/MDM2.f1 | SEQ ID NO: 190 | CTACAGGGACGCCATCGAA | 19 |
| MDM2 | NM_002392 | S0831/MDM2.r1 | SEQ ID NO: 191 | ATCCAACCAATCACCTGAATGTT | 23 |
| MDM2 | NM_002392 | S4834/MDM2.p1 | SEQ ID NO: 192 | CTTACACCAGCATCAAGATCCGG | 23 |
| MMP2 | NM_004530 | S1874/MMP2.f2 | SEQ ID NO: 193 | CCATGATGGAGAGGCAGACA | 20 |
| MMP2 | NM_004530 | S1875/MMP2.r2 | SEQ ID NO: 194 | GGAGTCCGTCCTTACCGTCAA | 21 |
| MMP2 | NM_004530 | S5039/MMP2.p2 | SEQ ID NO: 195 | CTGGGAGCATGGCGATGGATACCC | 24 |
| MMP9 | NM_004994 | S0656/MMP9.f1 | SEQ ID NO: 196 | GAGAACCAATCTCACCGACA | 20 |
| MMP9 | NM_004994 | S0657/MMP9.r1 | SEQ ID NO: 197 | CACCCGAGTGTAACCATAGC | 20 |
| MMP9 | NM_004994 | S4760/MMP9.p1 | SEQ ID NO: 198 | ACAGGTATTCCTCTGCCAGCTGCC | 24 |
| MVP | NM_017458 | S0193/MVP.f1 | SEQ ID NO: 199 | ACGAGAACGAGGGCATCTATGT | 22 |
| MVP | NM_017458 | S0195/MVP.r1 | SEQ ID NO: 200 | GCATGTAGGTGCTTCCAATCAC | 22 |
| MVP | NM_017458 | S5028/MVP.p1 | SEQ ID NO: 201 | CGCACCTTTCCGGTCTTGACATCCT | 25 |
| MYH11 | NM_002474 | S4555/MYH11.f1 | SEQ ID NO: 202 | CGGTACTTCTCAGGGCTAATATATACG | 27 |
| MYH11 | NM_002474 | S4556/MYH11.r1 | SEQ ID NO: 203 | CCGAGTAGATGGGCAGGTGTT | 21 |
| MYH11 | NM_002474 | S4557/MYH11.p1 | SEQ ID NO: 204 | CTCTTCTGCGTGGTGGTCAACCCCTA | 26 |
| NEK2 | NM_002497 | S4327/NEK2.f1 | SEQ ID NO: 205 | GTGAGGCAGCGCGACTCT | 18 |
| NEK2 | NM_002497 | S4328/NEK2.r1 | SEQ ID NO: 206 | TGCCAATGGTGTACAACACTTCA | 23 |
| NEK2 | NM_002497 | S4329/NEK2.p1 | SEQ ID NO: 207 | TGCCTTCCCGGGCTGAGGACT | 21 |
| NFKBp65 | NM_021975 | S0196/NFKBp6.f3 | SEQ ID NO: 208 | CTGCCGGGATGGCTTCTAT | 19 |
| NFKBp65 | NM_021975 | S0198/NFKBp6.r3 | SEQ ID NO: 209 | CCAGGTTCTGGAAACTGTGGAT | 22 |
| NFKBp65 | NM_021975 | S5030/NFKBp6.p3 | SEQ ID NO: 210 | CTGAGCTCTGCCCGGACCGCT | 21 |
| NPD009 | NM_020686 | S4474/NPD009.f3 | SEQ ID NO: 211 | GGCTGTGGCTGAGGCTGTAG | 20 |
| NPD009 | NM_020686 | S4475/NPD009.r3 | SEQ ID NO: 212 | GGAGCATTCGAGGTCAAATCA | 21 |
| NPD009 | NM_020686 | S4476/NPD009.p3 | SEQ ID NO: 213 | TTCCCAGAGTGTCTCACCTCCAGCAGAG | 28 |
| PDGFRb | NM_002609 | S1346/PDGFRb.f3 | SEQ ID NO: 214 | CCAGCTCTCCTTCCAGCTAC | 20 |
| PDGFRb | NM_002609 | S1347/PDGFRb.r3 | SEQ ID NO: 215 | GGGTGGCTCTCACTTAGCTC | 20 |
| PDGFRb | NM_002609 | S4931/PDGFRb.p3 | SEQ ID NO: 216 | ATCAATGTCCCTGTCCGAGTGCTG | 24 |
| PLAUR | NM_002659 | S1976/PLAUR.f3 | SEQ ID NO: 217 | CCCATGGATGCTCCTCTGAA | 20 |
| PLAUR | NM_002659 | S1977/PLAUR.r3 | SEQ ID NO: 218 | CCGGTGGCTACCAGACATTG | 20 |
| PLAUR | NM_002659 | S5054/PLAUR.p3 | SEQ ID NO: 219 | CATTGACTGCCGAGGCCCCATG | 22 |
| PR | NM_000926 | S1336/PR.f6 | SEQ ID NO: 220 | GCATCAGGCTGTCATTATGG | 20 |
| PR | NM_000926 | S1337/PR.r6 | SEQ ID NO: 221 | AGTAGTTGTGCTGCCCTTCC | 20 |
| PR | NM_000926 | S4743/PR.p6 | SEQ ID NO: 222 | TGTCCTTACCTGTGGGAGCTGTAAGGTC | 28 |
| pS2 | NM_003225 | S0241/pS2.f2 | SEQ ID NO: 223 | GCCCTCCCAGTGTGCAAAT | 19 |
| pS2 | NM_003225 | S0243/pS2.r2 | SEQ ID NO: 224 | CGTCGATGGTATTAGGATAGAAGCA | 25 |
| pS2 | NM_003225 | S5026/pS2.p2 | SEQ ID NO: 225 | TGCTGTTTCGACGACACCGTTCG | 23 |
| RAB27B | NM_004163 | S4336/RAB27B.f1 | SEQ ID NO: 226 | GGGACACTGCGGGACAAG | 18 |
| RAB27B | NM_004163 | S4337/RAB27B.r1 | SEQ ID NO: 227 | GCCCATGGCGTCTCTGAA | 18 |
| RAB27B | NM_004163 | S4338/RAB27B.p1 | SEQ ID NO: 228 | CGGTTCCGGAGTCTCACCACTGCAT | 25 |
| RAD54L | NM_003579 | S4369/RAD54L.f1 | SEQ ID NO: 229 | AGCTAGCCTCAGTGACACACATG | 23 |
| RAD54L | NM_003579 | S4370/RAD54L.r1 | SEQ ID NO: 230 | CCGGATCTGACGGCTGTT | 18 |
| RAD54L | NM_003579 | S4371/RAD54L.p1 | SEQ ID NO: 231 | ACACAACGTCGGCAGTGCAACCTG | 24 |
| RB1 | NM_000321 | S2700/RB1.f1 | SEQ ID NO: 232 | CGAAGCCCTTACAAGTTTCC | 20 |
| RB1 | NM_000321 | S2701/RB1.r1 | SEQ ID NO: 233 | GGACTCTTCAGGGGTGAAAT | 20 |
| RB1 | NM_000321 | S4765/RB1.p1 | SEQ ID NO: 234 | CCCTTACGGATTCCTGGAGGGAAC | 24 |
| RIZ1 | NM_012231 | S1320/RIZ1.f2 | SEQ ID NO: 235 | CCAGACGAGCGATTAGAAGC | 20 |
| RIZ1 | NM_012231 | S1321/RIZ1.r2 | SEQ ID NO: 236 | TCCTCCTCTTCCTCCTCCTC | 20 |
| RIZ1 | NM_012231 | S4761/R1Z1.p2 | SEQ ID NO: 237 | TGTGAGGTGAATGATTTGGGGGA | 23 |
| RPS6KB1 | NM_003161 | S2615/RPS6KB.f3 | SEQ ID NO: 238 | GCTCATTATGAAAAACATCCCAAAC | 25 |
| RPS6KB1 | NM_003161 | S2616/RPS6KB.r3 | SEQ ID NO: 239 | AAGAAACAGAAGTTGTCTGGCTTTCT | 26 |
| RPS6KB1 | NM_003161 | S4759/RPS6KB.p3 | SEQ ID NO: 240 | CACACCAACCAATAATTTCGCATT | 24 |
| STK15 | NM_003600 | S0794/STK15.f2 | SEQ ID NO: 241 | CATCTTCCAGGAGGACCACT | 20 |
| STK15 | NM_003600 | S0795/STK15.r2 | SEQ ID NO: 242 | TCCGACCTTCAATCATTTCA | 20 |
| STK15 | NM_003600 | S4745/STK15.p2 | SEQ ID NO: 243 | CTCTGTGGCACCCTGGACTACCTG | 24 |
| STMY3 | NM_005940 | S2067/STMY3.f3 | SEQ ID NO: 244 | CCTGGAGGCTGCAACATACC | 20 |
| STMY3 | NM_005940 | S2068/STMY3.r3 | SEQ ID NO: 245 | TACAATGGCTTTGGAGGATAGCA | 23 |
| STMY3 | NM_005940 | S4746/STMY3.p3 | SEQ ID NO: 246 | ATCCTCCTGAAGCCCTTTTCGCAGC | 25 |
| SURV | NM_001168 | S0259/SURV.f2 | SEQ ID NO: 247 | TGTTTTGATTCCCGGGCTTA | 20 |
| SURV | NM_001168 | S0261/SURV.r2 | SEQ ID NO: 248 | CAAAGCTGTCAGCTCTAGCAAAAG | 24 |
| SURV | NM_001168 | S4747/SURV.p2 | SEQ ID NO: 249 | TGCCTTCTTCCTCCCTCACTTCTCACCT | 28 |
| TGFB3 | NM_003239 | S1653/TGFB3.f1 | SEQ ID NO: 250 | GGATCGAGCTCTTCCAGATCCT | 22 |
| TGFB3 | NM_003239 | S1654/TGFB3.r1 | SEQ ID NO: 251 | GCCACCGATATAGCGCTGTT | 20 |
| TGFB3 | NM_003239 | S4911/TGFB3.p1 | SEQ ID NO: 252 | CGGCCAGATGAGCACATTGCC | 21 |
| TIMP2 | NM_003255 | S1680/TIMP2.f1 | SEQ ID NO: 253 | TCACCCTCTGTGACTTCATCGT | 22 |
| TIMP2 | NM_003255 | S1681/TIMP2.r1 | SEQ ID NO: 254 | TGTGGTTCAGGCTCTTCTTCTG | 22 |
| TIMP2 | NM_003255 | S4916/TIMP2.p1 | SEQ ID NO: 255 | CCCTGGGACACCCTGAGCACCA | 22 |
| TIMP3 | NM_000362 | S1641/TIMP3.f3 | SEQ ID NO: 256 | CTACCTGCCTTGCTTTGTGA | 20 |
| TIMP3 | NM_000362 | S1642/TIMP3.r3 | SEQ ID NO: 257 | ACCGAAATTGGAGAGCATGT | 20 |
| TIMP3 | NM_000362 | S4907/TIMP3.p3 | SEQ ID NO: 258 | CCAAGAACGAGTGTCTCTGGACCG | 24 |
| TOP2A | NM_001067 | S0271/TOP2A.f4 | SEQ ID NO: 259 | AATCCAAGGGGGAGAGTGAT | 20 |
| TOP2A | NM_001067 | S0273/TOP2A.r4 | SEQ ID NO: 260 | GTACAGATTTTGCCCGAGGA | 20 |
| TOP2A | NM_001067 | S4777/TOP2A.p4 | SEQ ID NO: 261 | CATATGGACTTTGACTCAGCTGTGGC | 26 |
| TP53BP1 | NM_005657 | S1747/TP53BP.f2 | SEQ ID NO: 262 | TGCTGTTGCTGAGTCTGTTG | 20 |
| TP53BP1 | NM_005657 | S1748/TP53BP.r2 | SEQ ID NO: 263 | CTTGCCTGGCTTCACAGATA | 20 |
| TP53BP1 | NM_005657 | S4924/TP53BP.p2 | SEQ ID NO: 264 | CCAGTCCCCAGAAGACCATGTCTG | 24 |
| VEGF | NM_003376 | S0286/VEGF.f1 | SEQ ID NO: 265 | CTGCTGTCTTGGGTGCATTG | 20 |
| VEGF | NM_003376 | S0288/VEGF.r1 | SEQ ID NO: 266 | GCAGCCTGGGACCACTTG | 18 |
| VEGF | NM_003376 | S4782/VEGF.p1 | SEQ ID NO: 267 | TTGCCTTGCTGCTCTACCTCCACCA | 25 |
| VEGFB | NM_003377 | S2724/VEGFB.f1 | SEQ ID NO: 268 | TGACGATGGCCTGGAGTGT | 19 |
| VEGFB | NM_003377 | S2725/VEGFB.r1 | SEQ ID NO: 269 | GGTACCGGATCATGAGGATCTG | 22 |
| VEGFB | NM_003377 | S4960/VEGFB.p1 | SEQ ID NO: 270 | CTGGGCAGCACCAAGTCCGGA | 21 |
| VEGFC | NM_005429 | S2251/VEGFC.f1 | SEQ ID NO: 271 | CCTCAGCAAGACGTTATTTGAAATT | 25 |
| VEGFC | NM_005429 | S2252/VEGFC.r1 | SEQ ID NO: 272 | AAGTGTGATTGGCAAAACTGATTG | 24 |
| VEGFC | NM_005429 | S4758/VEGFC.p1 | SEQ ID NO: 273 | CCTCTCTCTCAAGGCCCCAAACCAGT | 26 |
| VIM | NM_003380 | S0790/VIM.f3 | SEQ ID NO: 274 | TGCCCTTAAAGGAACCAATGA | 21 |
| VIM | NM_003380 | S0791/VIM.r3 | SEQ ID NO: 276 | GCTTCAACGGCAAAGTTCTCTT | 22 |
| VIM | NM_003380 | S4810/VIM.p3 | SEQ ID NO: 276 | ATTTCACGCATCTGGCGTTCCA | 22 |
| ZNF217 | NM_006526 | S2739/ZNF217.f3 | SEQ ID NO: 277 | ACCCAGTAGCAAGGAGAAGC | 20 |
| ZNF217 | NM_006526 | S2740/ZNF217.r3 | SEQ ID NO: 278 | CAGCTGGTGGTAGGTTCTGA | 20 |
| ZNF217 | NM_006526 | S4961/ZNF217.p3 | SEQ ID NO: 279 | CACTCACTGCTCCGAGTGCGG | 21 |

TABLE 4

| | | | | | |
|---|---|---|---|---|---|
| Name | Accession Number | Version | Gene Sequence Start | Gene Sequence Stop | SEQ ID Nos. |

| | | | | | |
|---|---|---|---|---|---|
| ACTG2 | NM_001615 | 3 | 477 | 560 | SEQ ID NO: 280 |
| AKT1 | NM_005163 | 3 | 949 | 1020 | SEQ ID NO: 281 |
| B-Catenin | NM_001904 | 3 | 1549 | 1629 | SEQ ID NO: 282 |
| BAD | NM_032989 | 1 | 34 | 107 | SEQ ID NO: 283 |
| BBC3 | NM_014417 | 2 | 500 | 583 | SEQ ID NO: 284 |
| Bcl2 | NM_000633 | 2 | 1386 | 1459 | SEQ ID NO: 285 |
| Bclx | NM_001191 | 2 | 514 | 584 | SEQ ID NO: 286 |
| BECN1 | NM_003766 | 3 | 974 | 1051 | SEQ ID NO: 287 |
| BIN1 | NM_004305 | 3 | 866 | 942 | SEQ ID NO: 288 |
| BRK | NM_005975 | 2 | 2279 | 2358 | SEQ ID NO: 289 |
| C20 orf1 | NM_012112 | 1 | 2675 | 2740 | SEQ ID NO: 290 |
| CA9 | NM_001216 | 3 | 1285 | 1357 | SEQ ID NO: 291 |
| CCNB1 | NM_031966 | 2 | 823 | 907 | SEQ ID NO: 292 |
| CCND1 | NM_001758 | 3 | 461 | 530 | SEQ ID NO: 293 |
| CD31 | NM_000442 | 3 | 2422 | 2497 | SEQ ID NO: 294 |
| CD3z | NM_000734 | 1 | 177 | 242 | SEQ ID NO: 295 |
| CD9 | NM_001769 | 1 | 522 | 586 | SEQ ID NO: 296 |
| CDC20 | NM_001255 | 1 | 679 | 747 | SEQ ID NO: 297 |
| CDH1 | NM_004360 | 3 | 2499 | 2580 | SEQ ID NO: 298 |
| CEGP1 | NM_020974 | 2 | 563 | 640 | SEQ ID NO: 299 |
| Chk2 | NM_007194 | 3 | 1152 | 1230 | SEQ ID NO: 300 |
| cIAP2 | NM_001165 | 2 | 1118 | 1204 | SEQ ID NO: 301 |
| cMet | NM_000245 | 2 | 1750 | 1836 | SEQ ID NO: 302 |
| CNN | NM_001299 | 1 | 533 | 597 | SEQ ID NO: 303 |
| COL1A1 | NM_000088 | 1 | 4161 | 4229 | SEQ ID NO: 304 |
| COL1A2 | NM_000089 | 1 | 3772 | 3852 | SEQ ID NO: 305 |
| COX2 | NM_000963 | 1 | 1554 | 1633 | SEQ ID NO: 306 |
| CTSL2 | NM_001333 | 1 | 671 | 738 | SEQ ID NO: 307 |
| CYP2C8 | NM_000770 | 2 | 452 | 525 | SEQ ID NO: 308 |
| DHPS | NM_013407 | 3 | 573 | 651 | SEQ ID NO: 309 |
| DIABLO | NM_019887 | 1 | 16 | 89 | SEQ ID NO: 310 |
| DKFZp564 | XM_047080 | 2 | 1689 | 1764 | SEQ ID NO: 311 |
| DR5 | NM_003842 | 2 | 1127 | 1211 | SEQ ID NO: 312 |
| EGFR | NM_005228 | 2 | 713 | 775 | SEQ ID NO: 313 |
| EIF4EL3 | NM_004846 | 1 | 729 | 796 | SEQ ID NO: 314 |
| EPHX1 | NM_000120 | 2 | 1200 | 1276 | SEQ ID NO: 315 |
| ErbB3 | NM_001982 | 1 | 3669 | 3750 | SEQ ID NO: 316 |
| EstR1 | NM_000125 | 1 | 1956 | 2024 | SEQ ID NO: 317 |
| FGFR1 | NM_023109 | 3 | 2685 | 2759 | SEQ ID NO: 318 |
| FLJ20354 | NM_017779 | 1 | 1946 | 2019 | SEQ ID NO: 319 |
| G-Catenin | NM_002230 | 1 | 229 | 297 | SEQ ID NO: 320 |
| GATA3 | NM_002051 | 3 | 1630 | 1705 | SEQ ID NO: 321 |
| GSN | NM_000177 | 3 | 2188 | 2273 | SEQ ID NO: 322 |
| GSTp | NM_000852 | 3 | 420 | 496 | SEQ ID NO: 323 |
| HER2 | NM_004448 | 3 | 1138 | 1208 | SEQ ID NO: 324 |
| HIF1A | NM_001530 | 3 | 809 | 891 | SEQ ID NO: 325 |
| HLA-DPB1 | NM_002121 | 1 | 57 | 130 | SEQ ID NO: 326 |
| HNF3A | NM_004496 | 1 | 82 | 155 | SEQ ID NO: 327 |
| ID1 | NM_002165 | 1 | 286 | 356 | SEQ ID NO: 328 |
| ID2 | NM_002166 | 4 | 226 | 302 | SEQ ID NO: 329 |
| IGF1R | NM_000875 | 3 | 3467 | 3550 | SEQ ID NO: 330 |
| IGFBP2 | NM_000597 | 1 | 613 | 686 | SEQ ID NO: 331 |
| IRS1 | NM_005544 | 3 | 3765 | 3839 | SEQ ID NO: 332 |
| KI-67 | NM_002417 | 2 | 42 | 122 | SEQ ID NO: 333 |
| KIAA1209 | AJ420468 | 1 | 1089 | 1160 | SEQ ID NO: 334 |
| KLK10 | NM_002776 | 3 | 966 | 1044 | SEQ ID NO: 335 |
| KRT14 | NM_000526 | 1 | 525 | 608 | SEQ ID NO: 338 |
| KRT17 | NM_000422 | 2 | 861 | 934 | SEQ ID NO: 337 |
| KRT18 | NM_000224 | 2 | 654 | 722 | SEQ ID NO: 338 |
| KRT19 | NM_002276 | 3 | 1100 | 1177 | SEQ ID NO: 339 |
| KRT5 | NM_000424 | 3 | 1605 | 1674 | SEQ ID NO: 340 |
| MCM2 | NM_004526 | 2 | 2442 | 2517 | SEQ ID NO: 341 |
| MCM3 | NM_002388 | 3 | 581 | 656 | SEQ ID NO: 342 |
| MDM2 | NM_002392 | 1 | 955 | 1023 | SEQ ID NO: 343 |
| MMP2 | NM_004530 | 2 | 775 | 861 | SEQ ID NO: 344 |
| MMP9 | NM_004994 | 1 | 124 | 191 | SEQ ID NO: 345 |
| MVP | NM_017458 | 1 | 1268 | 1343 | SEQ ID NO: 346 |
| MYH11 | NM_002474 | 1 | 410 | 495 | SEQ ID NO: 347 |
| NEK2 | NM_002497 | 1 | 102 | 181 | SEQ ID NO: 348 |
| NFKBp65 | NM_021975 | 3 | 281 | 349 | SEQ ID NO: 349 |
| NPD009 | NM_020686 | 3 | 589 | 662 | SEQ ID NO: 350 |
| PDGFRb | NM_002609 | 3 | 1571 | 1637 | SEQ ID NO: 351 |
| PLAUR | NM_002659 | 3 | 1097 | 1173 | SEQ ID NO: 352 |
| PR | NM_000926 | 6 | 1895 | 1980 | SEQ ID NO: 353 |
| pS2 | NM_003225 | 2 | 181 | 267 | SEQ ID NO: 354 |
| RAB27B | NM_004163 | 1 | 329 | 394 | SEQ ID NO: 355 |
| RAD54L | NM_003579 | 1 | 2668 | 2735 | SEQ ID NO: 356 |
| RB1 | NM_000321 | 1 | 2497 | 2574 | SEQ ID NO: 357 |
| RIZ1 | NM_012231 | 2 | 1609 | 1683 | SEQ ID NO: 358 |
| RPS6KB1 | NM_003161 | 3 | 2155 | 2236 | SEQ ID NO: 359 |
| STK15 | NM_003600 | 2 | 1101 | 1170 | SEQ ID NO: 360 |
| STMY3 | NM_005940 | 3 | 2090 | 2180 | SEQ ID NO: 361 |
| SURV | NM_001168 | 2 | 737 | 817 | SEQ ID NO: 362 |
| TGFB3 | NM_003239 | 1 | 753 | 818 | SEQ ID NO: 383 |
| TIMP2 | NM_003255 | 1 | 673 | 742 | SEQ ID NO: 364 |
| TIMP3 | NM_000362 | 3 | 1636 | 1703 | SEQ ID NO: 365 |
| TOP2A | NM_001067 | 4 | 4505 | 4577 | SEQ ID NO: 366 |
| TP53BP1 | NM_005657 | 2 | 3233 | 3307 | SEQ ID NO: 367 |
| VEGF | NM_003376 | 1 | 26 | 97 | SEQ ID NO: 368 |
| VEGFB | NM_003377 | 1 | 298 | 369 | SEQ ID NO: 369 |
| VEGFC | NM_005429 | 1 | 970 | 1053 | SEQ ID NO: 370 |
| VIM | NM_003380 | 3 | 1115 | 1187 | SEQ ID NO: 371 |
| ZNF217 | NM_006526 | 3 | 1372 | 1442 | SEQ ID NO: 372 |

| | |
|---|---|
| Name | Sequence |

| | |
|---|---|
| ACTG2 | ATGTACGTCGCCATTCAAGCTGTGCTCTCCCTCTATGCCTCTGGCCGCACGACAGGCATCGTCCTGGATTCAGGTGATGGCGT |
| AKT1 | CGCTTCTATGGCGCTGAGATTGTGTCAGCCCTGGACTACCTGCACTCGGAGAAGAACGTGGTGTACCGGGA |
| B-Catenin | GGCTCTTGTGCGTACTGTCCTTCGGGCTGGTGACAGGGAAGACATCACTGAGCCTGCCATCTGTGCTCTTCGTCATCTGA |
| BAD | GGGTCAGGTGCCTCGAGATCGGGCTTGGGCCCAGAGCATGTTCCAGATCCCAGAGTTTGAGCCGAGTGAGCAG |
| BBC3 | CCTGGAGGGTCCTGTACAATCTCATCATGGGACTCCTGCCCTTACCCAGGGGCCACAGAGCCCCCGAGATGGAGCCCAATTAG |
| Bcl2 | CAGATGGACCTAGTACCCACTGAGATTTCCACGCCGAAGGACAGCGATGGGAAAAATGCCCTTAAATCATAGG |
| Bclx | CTTTTGTGGAACTCTATGGGAACAATGCAGCAGCCGAGAGCCGAAAGGGCCAGGAACGCTTCAACCGCTG |
| BECN1 | CAGTTTGGCACAATCAATAACTTCAGGCTGGGTCGCCTGCCCAGTGTTCCCGTGGAATGGAATGAGATTAATGCTGC |
| BIN1 | CCTGCAAAAGGGAACAAGAGCCCTTCGCCTCCAGATGGCTCCCCTGCCGCCACCCCCGAGATCAGAGTCAACCACG |
| BRK | GTGCAGGAAAGGTTCACAAATGTGGAGTGTCTGCGTCCAATACACGCGTGTGCTCCTCTCCTTACTCCATCGTGTGTGC |
| C20 orf1 | TCAGCTGTGAGCTGCGGATACCGCCCGGCAATGGGACCTGCTCTTAACCTCAAACCTAGGACCGT |
| CA9 | ATCCTAGCCCTGGTTTTTGGCCTCCTTTTTGCTGTCACCAGCGTCGCGTTCCTTGTGCAGATGAGAAGGCAG |
| CCNB1 | TTCAGGTTGTTGCAGGAGACCATGTACATGACTGTCTCTATTATTGATCGGTTCATGCAGAATAATTGTGTGCCCAAGAAGATG |
| CCND1 | GCATGTTCGTGGCCTCTAAGATGAAGGAGACCATCCCCCTGACGGCCGAGAAGCTGTGCATCTACACCG |
| CD31 | TGTATTTCAAGACCTCTGTGCACTTATTTATGAACCTGCCCTGCTCCCACAGAACACAGCAATTCCTCAGGCTAA |
| CD3z | AGATGAAGTGGAAGGCGCTTTTCACCGCGGCCATCCTGCAGGCACAGTTGCCGATTACAGAGGCA |
| CD9 | GGGCGTGGAACAGTTTATCTCAGACATCTGCCCCAAGAAGGACGTACTCGAAACCTTCACCGTG |
| CDC20 | TGGATTGGAGTTCTGGGAATGTACTGGCCGTGGCACTGGACAACAGTGTGTACCTGTGGAGTGCAAGC |
| CDH1 | TGAGTGTCCCCCGGTATCTTCCCCGCCCTGCCAATCCCGATGAAATTGGAAATTTTATTGATGAAAATCTGAAAGCGGCTG |
| CEGP1 | TGACAATCAGCACACCTGCATTCACCGCTCGGAAGAGGGCCTGAGCTGCATGAATAAGGATCACGGCTGTAGTCACA |
| Chk2 | ATGTGGAACCCCCACCTACTTGGCGCCTGAAGTTCTTGTTTCTGTTGGGACTGCTGGGTATAACCGTGCTGTGGACTG |
| cIAP2 | GGATATTTCCGTGGCTCTTATTCAAACTCTCCATCAAATCCTGTAAACTCCAGAGCAAATCAAGATTTTTCTGCCTTGATGAGAAG |
| cMet | GACATTTCCAGTCCTGCAGTCAATGCCTCTCTGCCCCACCCTTTGTTCAGTGTGGCTGGTGCCACGACAAATGTGTGCGATCGGAG |
| CNN | TCCACCCTCCTGGCTTTGGCCAGCATGGCGAAGACGAAAGGAAACAAGGTGAACGTGGGAGTGA |
| COL1A1 | GTGGCCATCCAGCTGACCTTCCTGCGCCTGATGTCCACCGAGGCCTCCCAGAACATCACCTACCACTG |
| COL1A2 | CAGCCAAGAACTGGTATAGGAGCTCCAAGGAGAAGAAACACGTCTGGCTAGGAGAAACTATCAATGCTGGCAGCCAGTTT |
| COX2 | TCTGCAGAGTTGGAAGCACTCTATGGTGACATCGATGCTGTGGAGCTGTATCCTGCCCTTCTGGTAGAAAAGCCTCGGC |
| CTSL2 | TGTCTCACTGAGCGAGCAGAATCTGGTGGACTGTTCGCGTCCTCAAGGCAATCAGGGCTGCAATGGT |
| CYP2C8 | CCGTGTTCAAGAGGAAGCTCACTGCCTTGTGGAGGAGTTGAGAAAAACCAAGGCTTCACCCTGTGATCCCACT |
| DHPS | GGGAGAACGGGATCAATAGGATCGGAAACCTGCTGGTGCCCAATGAGAATTACTGCAAGTTTGAGGACTGGCTGATGC |
| DIABLO | CACAATGGCGGCTCTGAAGAGTTGGCTGTCGCGCAGCGTAACTTCATTCTTCAGGTACAGACAGTGTTTGTGT |
| DKFZp564 | CAGTGCTTCCATGGACAAGTCCTTGTCAAAACTGGCCCATGCTGATGGAGATCAAACATCAATCATCCCTGTCCA |
| DR5 | CTCTGAGACAGTGCTTCGATGACTTTGCAGACTTGGTGCCCTTTGACTCCTGGGAGCCGCTCATGAGGAAGTTGGGCCTCATGG |
| EGFR | TGTCGATGGACTTCCAGAACCACCTGGGCAGCTGCCAAAAGTGTGATCCAAGCTGTCCCAAT |
| EIF4EL3 | AAGCCGCGGTTGAATGTGCCATGACCCTCTCCCTCTCTGGATGGCACCATCATTGAAGCTGGCGTCA |
| EPHX1 | ACCGTAGGCTCTGCTCTGAATGACTCTCCTGTGGGTCTGGCTGCCTATATTCTAGAGAAGTTTTCCACCTGGACCA |
| ErbB3 | CGGTTATGTCATGCCAGATACACACCTCAAAGGTACTCCCTCCTCCCGGGAAGGCACCCTTTCTTCAGTGGGTCTCAGTTC |
| EstR1 | CGTGGTGCCCCTCTATGACCTGCTGCTGGAGATGCTGGACGCCCACCGCCTACATGCGCCCACTAGCC |
| FGFR1 | CACGGGACATTCACCACATCGACTACTATAAAAAGACAACCAACGGCCGACTGCCTGTGAAGTGGATGGCACCC |
| FLJ20354 | GCGTATGATTTCCCGAATGAGTCAAAATGTTGATATGCCCAAACTTCATGATGCAATGGGTACGAGGTCACTG |
| G-Catenin | TCAGCAGCAAGGGCATCATGGAGGAGGATGAGGCCTGCGGGCGCCAGTACACGCTCAAGAAAACCACC |
| GATA3 | CAAAGGAGCTCACTGTGGTGTCTGTGTTCCAACCACTGAATCTGGACCCCATCTGTGAATAAGCCATTCTGACTC |
| GSN | CTTCTGCTAAGCGGTACATCGAGACGGACCCAGCCAATCGGGATCGGCGGACGCCCATCACCGTGGTGAAGCAAGGCTTTGAGCC |
| GSTp | GAGACCCTGCTGTCCCAGAACCAGGGAGGCAAGACCTTCATTGTGGGAGACCAGATCTCCTTCGCTGACTACAACC |
| HER2 | CGGTGTGAGAAGTGCAGCAAGCCCTGTGCCCGAGTGTGCTATGGTCTGGGCATGGAGCACTTGCGAGAGG |
| HIF1A | TGAACATAAAGTCTGCAACATGGAAGGTATTGCACTGCACAGGCCACATTCACGTATATGATACCAACAGTAACCAACCTCA |
| HLA-DPB1 | TCCATGATGGTTCTGCAGGTTTCTGCGGCCCCCCGGACAGTGGCTCTGACGGCGTTACTGATGGTGCTGCTCA |
| HNF3A | TCCAGGATGTTAGGAACTGTGAAGATGGAAGGGCATGAAACCAGCGACTGGAACAGCTACTACGCAGACACGC |
| ID1 | AGAACCGCAAGGTGAGCAAGGTGGAGATTCTCCAGCACGTCATCGACTACATCAGGGACCTTCAGTTGGA |
| ID2 | AACGACTGCTACTCCAAGCTCAAGGAGCTGGTGCCCAGCATCCCCCAGAACAAGAAGGTGAGCAAGATGGAAATCC |
| IGF1R | GCATGGTAGCCGAAGATTTCACAGTCAAAATCGGAGATTTTGGTATGACGCGAGATATCTATGAGACAGACTATTACCGGAAA |
| IGFBP2 | GTGGACAGCACCATGAACATGTTGGGCGGGGGAGGCAGTGCTGGCCGGAAGCCCCTCAAGTCGGGTATGAAGG |
| IRS1 | CCACAGCTCACCTTCTGTCAGGTGTCCATCCCAGCTCCAGCCAGCTCCCAGAGAGGAAGAGACTGGCACTGAGG |
| KI-67 | CGGACTTTGGGTGCGACTTGACGAGCGGTGGTTCGACAAGTGGCCTTGCGGGCCGGATCGTCCCAGTGGAAGAGTTGTAA |
| KIAA1209 | GCCTAGCAGTTCTACCATGATCAGCGTGCTTCGAGCGGGTGGAGCTCTCAGAAACATCTGGACCGATCACC |
| KLK10 | GCCCAGAGGCTCCATCGTCCATCCTCTTCCTCCCCAGTCGGCTGAACTCTCCCCTTGTCTGCACTGTTCAAACCTCTG |
| KRT14 | GGCCTGCTGAGATCAAAGACTACAGTCCCTACTTCAAGACCATTGAGGACCTGAGGAACAAGATTCTCACAGCCACAGTGGAC |
| KRT17 | CGAGGATTGGTTCTTCAGCAAGACAGAGGAACTGAACCGCGAGGTGGCCACCAACAGTGAGCTGGTGCAGAGT |
| KRT18 | AGAGATCGAGGCTCTCAAGGAGGAGCTGCTCTTCATGAAGAAGAACCACGAAGAGGAAGTAAAAGGCC |
| KRT19 | TGAGCGGCAGAATCAGGAGTACCAGCGGCTCATGGACATCAAGTCGCGGCTGGAGCAGGAGATTGCCACCTACCGCA |
| KRT5 | TCAGTGGAGAAGGAGTTGGACCAGTCAACATCTCTGTTGTCACAAGCAGTGTTTCCTCTGGATATGGCA |
| MCM2 | GACTTTTGCCCGCTACCTTTCATTCCGGCGTGACAACAATGAGCTGTTGCTCTTCATACTGAAGCAGTTAGTGGC |
| MCM3 | GGAGAACAATCCCCTTGAGACAGAATATGGCCTTTCTGTCTACAAGGATCACCAGACCATCACCATCCAGGAGAT |
| MDM2 | CTACAGGGACGCCATCGAATCCGGATCTTGATGCTGGTGTAAGTGAACATTCAGGTGATTGGTTGGAT |
| MMP2 | CCATGATGGAGAGGCAGACATCATGATCAACTTTGGCCGCTGGGAGCATGGCGATGGATACCCCTTTGACGGTAAGGACGGACTCC |
| MMP9 | GAGAACCAATCTCACCGACAGGCAGCTGGCAGAGGAATACCTGTACCGCTATGGTTACACTCGGGTG |
| MVP | ACGAGAACGAGGGCATCTATGTGCAGGATGTCAAGACCGGAAAGGTGCGCGCTGTGATTGGAAGCACCTACATGC |
| MYH11 | CGGTACTTCTCAGGGCTAATATATACGTACTCTGGCCTCTTCTGCGTGGTGGTCAACCCCTATAAACACCTGCCCATCTACTCGG |
| NEK2 | GTGAGGCAGCGCGACTCTGGCGACTGGCCGGCCATGCCTTCCCGGGCTGAGGACTATGAAGTGTTGTACACCATTGGCA |
| NFKBp65 | CTGCCGGGATGGCTTCTATGAGGCTGAGCTCTGCCCGGACCGCTGCATCCACAGTTTCCAGAACCTGG |
| NPD009 | GGCTGTGGCTGAGGCTGTAGCATCTCTGCTGGAGGTGAGACACTCTGGGAACTGATTTGACCTCGAATGCTCC |
| PDGFRb | CCAGCTCTCCTTCCAGCTACAGATCAATGTCCCTGTCCGAGTGCTGGAGCTAAGTGAGAGCCACCC |
| PLAUR | CCCATGGATGCTCCTCTGAAGAGACTTTCCTCATTGACTGCCGAGGCCCCATGAATCAATGTCTGGTAGCCACCGG |
| PR | GCATCAGGCTGTCATTATGGTGTCCTTACCTGTGGGAGCTGTAAGGTCTTCTTTAAGAGGGCAATGGAAGGGCAGCACAACTACT |
| pS2 | GCCCTCCCAGTGTGCAAATAAGGGCTGCTGTTTCGACGACACCGTTCGTGGGGTCCCCTGGTGCTTCTATCCTAATACCATCGACG |
| RAB27B | GGGACACTGCGGGACAAGAGCGGTTCCGGAGTCTCACCACTGCATTTTTCAGAGACGCCATGGGC |
| RAD54L | AGCTAGCCTCAGTGACACACATGACAGGTTGCACTGCCGACGTTGTGTCAACAGCCGTCAGATCCGG |
| RB1 | CGAAGCCCTTACAAGTTTCCTAGTTCACCCTTACGGATTCCTGGAGGGAACATCTATATTTCACCCCTGAAGAGTCC |
| RIZ1 | CCAGACGAGCGATTAGAAGCGGCAGCTTGTGAGGTGAATGATTTGGGGGAAGAGGAGGAGGAGGAAGAGGAGGA |
| RPS6KB1 | GCTCATTATGAAAAACATCCCAAACTTTAAAATGCGAAATTATTGGTTGGTGTGAAGAAAGCCAGACAACTTCTGTTTCTT |
| STK15 | CATCTTCCAGGAGGACCACTCTCTGTGGCACCCTGGACTACCTGCCCCCTGAAATGATTGAAGGTCGGA |
| STMY3 | CCTGGAGGCTGCAACATACCTCAATCCTGTCCCAGGCCGGATCCTCCTGAAGCCCTTTTCGCAGCACTGCTATCCTCCAAAGCCATTGTA |
| SURV | TGTTTTGATTCCCGGGCTTACCAGGTGAGAAGTGAGGGAGGAAGAAGGCAGTGTCCCTTTTGCTAGAGCTGACAGCTTTG |
| TGFB3 | GGATCGAGCTCTTCCAGATCCTTCGGCCAGATGAGCACATTGCCAAACAGCGCTATATCGGTGGC |
| TIMP2 | TCACCCTCTGTGACTTCATCGTGCCCTGGGACACCCTGAGCACCACCCAGAAGAAGAGCCTGAACCACA |
| TIMP3 | CTACCTGCCTTGCTTTGTGACTTCCAAGAACGAGTGTCTCTGGACCGACATGCTCTCCAATTTCGGT |
| TOP2A | AATCCAAGGGGGAGAGTGATGACTTCCATATGGACTTTGACTCAGCTGTGGCTCCTCGGGCAAAATCTGTAC |
| TP53BP1 | TGCTGTTGCTGAGTCTGTTGCCAGTCCCCAGAAGACCATGTCTGTGTTGAGCTGTATCTGTGAAGCCAGGCAAG |
| VEGF | CTGCTGTCTTGGGTGCATTGGAGCCTTGCCTTGCTGCTCTACCTCCACCATGCCAAGTGGTCCCAGGCTGC |
| VEGFB | TGACGATGGCCTGGAGTGTGTGCCCACTGGGCAGCACCAAGTCCGGATGCAGATCCTCATGATCCGGTACC |
| VEGFC | CCTCAGCAAGACGTTATTTGAAATTACAGTGCCTCTCTCTCAAGGCCCCAAACCAGTAACAATCAGTTTTGCCAATCACACTT |
| VIM | TGCCCTTAAAGGAACCAATGAGTCCCTGGAACGCCAGATGCGTGAAATGGAAGAGAACTTTGCCGTTGAAGC |
| ZNF217 | ACCCAGTAGCAAGGAGAAGCCCACTCACTGCTCCGAGTGCGGCAAAGCTTTCAGAACCTACCACCAGCTG |

1. A method for predicting the response of a subject diagnosed with cancer to chemotherapy comprising:
determining the expression level of one or more prognostic RNA transcripts or their expression products in a biological sample comprising cancer cells obtained from said subject, wherein the prognostic RNA transcript is the transcript of one or more genes selected from the group consisting of VEGFC; B-Catenin; MMP2; MMP9; CNN; FLJ20354; TGFB3; PDGFRb; PLAUR; KRT19; ID1; RIZ1; RAD54L; RB1; SURV; EIF4EL3; CYP2C8; STK15; ACTG2; NEK2; cMet; TIMP2; C20 orf1; DR5; CD31; BIN1; COL1A2; HIF1A; VIM; CDC20; ID2; MCM2; CCNB1; MYH11; Chk2; G-Catenin; HER2; GSN; Ki-67; TOP2A; CCND1; EstR1; KRT18; GATA3; cIAP2; KRT5; RAB27B; IGF1R; HNF3A; CA9; MCM3; STMY3; NPD009; BAD; BBC3; EGFR; CD9; AKT1; CD3z; KRT14; DKFZp564; Bcl2; BECN1; KLK10; DIABLO; MVP; VEGFB; ErbB3; MDM2; Bclx; CDH1; HLA-DPB1; PR; KRT17; GSTp; IRS1; NFKBp65; IGFBP2; RPS6 KB1; DHPS; TIMP3; ZNF217; KIAA1209; COX2; pS2; BRK; CEGP1; EPHX1; VEGF; TP53BP1; COL1A1; FGFR1; and CTSL2, wherein
(a) for every unit of increased expression of one or more of MMP9; FLJ20354; RAD54L; SURV; CYP2C8; STK15; NEK2; C20 orf1; CDC20; MCM2; CCNB1; Chk2; Ki-67; TOP2A; CCND1; EstR1; KRT18; GATA3; RAB27B; IGF1R; HNF3A; STMY3; NPD009; BAD; BBC3; CD9; AKT1; Bcl2; BECN1; DIABLO; MVP; VEGFB; ErbB3; MDM2; Bclx; CDH1; PR; IRS1; NFKBp65; IGFBP2; RPS6 KB1; DHPS; TIMP3; ZNF217; pS2; BRK; CEGP1; EPHX1; TP53BP1; COL1A1; and FGFR1, or the corresponding expression product, said subject is predicted to have an increased likelihood of response; and
(b) for every unit of increased expression of one or more of VEGFC; B-Catenin; MMP2; CNN; TGFB3; PDGFRb; PLAUR; KRT19; ID1; RIZ1; RB1; EIF4EL3; ACTG2; cMet; TIMP2; DR5; CD31; BIN1; COL1A2; HIF1A; VIM; ID2; MYH11; G-Catenin; HER2; GSN; cIAP2; KRT5; CA9; MCM3; EGFR; CD3z; KRT14; DKFZp564; KLK10; HLA-DPB1; KRT17; GSTp; KIAA1209; COX2; VEGF; and CTSL2, or the corresponding expression product, said subject is predicted to have a decreased likelihood of response.

2. The method of claim 1 wherein said response is clinical response.

3. The method of claim 2 wherein the prognostic RNA transcript is the transcript of one or more genes selected from the group consisting of CCND1; EstR1; KRT18; GATA3; cIAP2; KRT5; RAB27B; IGF1R; HNF3A; CA9; MCM3; STMY3; NPD009; BAD; BBC3; EGFR; CD9; AKT1; CD3z; KRT14; DKFZp564; Bcl2; BECN1; KLK10; DIABLO; MVP; VEGFB; ErbB3; MDM2; Bclx; CDH1; HLA-DPB1; PR; KRT17; GSTp; IRS1; NFKBp65; IGFBP2; RPS6 KB1; DHPS; TIMP3; ZNF217; KIAA1209; COX2; pS2; BRK; CEGP1; EPHX1; VEGF; TP53BP1; COL1A1; FGFR1; and CTSL2; wherein
(a) for every unit of increased expression of one or more of CCND1; EstR1; KRT18; GATA3; RAB27B; IGF1R; HNF3A; STMY3; NPD009; BAD; BBC3; CD9; AKT1; Bcl2; BECN1; DIABLO; MVP; VEGFB; ErbB3; MDM2; Bclx; CDH1; PR; IRS1; NFKBp65; IGFBP2; RPS6 KB1; DHPS; TIMP3; ZNF217; pS2; BRK; CEGP1; EPHX1; TP53BP1; COL1A1; and FGFR1, or the corresponding expression products said subject is predicted to have an increased likelihood of response; and
(b) for every unit of increased expression of one or more of cIAP2; KRT5; CA9; MCM3; EGFR; CD3z; KRT14; DKFZp564; KLK10; HLA-DPB1; KRT17; GSTp; KIAA1209; COX2; VEGF; and CTSL2, or the corresponding expression products said subject is predicted to have a decreased likelihood of response.

4. The method of claim 1 wherein said response is pathogenic response.

5. The method of claim 4 wherein the prognostic RNA transcript is the transcript of one or more genes selected from the group consisting of VEGFC; B-Catenin; MMP2; MMP9; CNN; FLJ20354; TGFB3; PDGFRb; PLAUR; KRT19; ID1; RIZ1; RAD54L; RB1; SURV; EIF4EL3; CYP2C8; STK15; ACTG2; NEK2; cMet; TIMP2; C20 orf1; DR5; CD31; BIN1; COL1A2; HIF1A; VIM; CDC20; ID2; MCM2; CCNB1; MYH11; Chk2; G-Catenin; HER2; GSN; Ki-67; TOP2A; and
(a) for every unit of increased expression of one or more of MMP9; FLJ20354; RAD54L; SURV; CYP2C8; STK15; NEK2; C20 orf1; CDC20; MCM2; CCNB1; Chk2; Ki-67; TOP2A, or the corresponding expression products said subject is predicted to have an increased likelihood of response; and
(b) for every unit of increased expression of one or more of VEGFC; B-Catenin; MMP2; CNN; TGFB3; PDGFRb; PLAUR; KRT19; ID1; RIZ1; RB1; EIF4EL3; ACTG2; cMet; TIMP2; DR5; CD31; BIN1; COL1A2; HIF1A; VIM; ID2; MYH11; G-Catenin; HER2; GSN, or the corresponding expression products said subject is predicted to have a decreased likelihood of response.

6. The method of claim 1 wherein said subject is a human patient.

7. The method of claim 6 wherein said cancer is selected from the group consisting of breast cancer, ovarian cancer, gastric cancer, colorectal cancer, prostate cancer; pancreatic cancer, and lung cancer.

8. The method of claim 7 wherein said cancer is breast cancer.

9. The method of claim 8 wherein said cancer is invasive breast cancer.

10. The method of claim 9 wherein said cancer is stage II or stage III breast cancer.

11. The method of claim 9 wherein said chemotherapy is neoadjuvant chemotherapy.

12. The method of claim 8 wherein said chemotherapy comprises the administration of a taxane derivative.

13. The method of claim 12 wherein said taxane is docetaxel or paclitaxel.

14. The method of claim 13 wherein said taxane is docetaxel.

15. The method of claim 8 wherein said chemotherapy comprises the administration of an anthracycline derivative.

16. The method of claim 15 wherein said anthracycline derivative is doxorubicin.

17. The method of claim 8 wherein said chemotherapy comprises the administration of a topoisomerase inhibitor.

18. The method of claim 17 wherein said topoisomerase inhibitor is selected from the group consisting of camptothecin, topotecan, irinotecan, 20-S-camptothecin, 9-nitro-camptothecin, 9-amino-camptothecin, and GI147211.

19. The method of claim 8 wherein said chemotherapy comprises the administration of at least two chemotherapeutic agents.

20. The method of claim 19 wherein said chemotherapeutic agents are selected from the group consisting of taxane derivatives, anthracycline derivatives and topoisomerase inhibitors.

21. The method of claim 1 comprising determining the expression level of at least two of said prognostic transcripts or their expression products.

22. The method of claim 1 comprising determining the expression level of at least five of said prognostic transcripts or their expression products.

23. The method of claim 1 comprising determining the expression level of all of said prognostic transcripts or their expression products.

24. The method of claim 1 wherein said biological sample is a tissue sample comprising cancer cells.

25. The method of claim 24 wherein said tissue is fixed, paraffin-embedded, or fresh, or frozen.

26 The method of claim 24 where the tissue is from fine needle, core, or other types of biopsy.

27. The method of claim 24 wherein the tissue sample is obtained by fine needle aspiration, bronchial lavage, or transbronchial biopsy.

28. The method of claim 1 wherein the expression level of said prognostic RNA transcript or transcripts is determined by RT-PCR.

29. The method of claim 1 wherein the expression level of said expression product or products is determined by immunohistochemistry.

30. The method of claim 1 wherein the expression level of said expression product or products is determined by proteomics techniques.

31. The method of claim 1 wherein the assay for the measurement of said prognostic RNA transcripts or their expression products is provided is provided in the form of a kit or kits.

32. An array comprising polynucleotides hybridizing to one or more of the following genes: VEGFC; B-Catenin; MMP2; MMP9; CNN; FLJ20354; TGFB3; PDGFRb; PLAUR; KRT19; ID1; RIZ1; RAD54L; RB1; SURV; EIF4EL3; CYP2C8; STK15; ACTG2; NEK2; cMet; TIMP2; C20 orf1; DR5; CD31; BIN1; COL1A2; HIF1A; VIM; CDC20; ID2; MCM2; CCNB1; MYH11; Chk2; G-Catenin; HER2; GSN; Ki-67; TOP2A; CCND1; EstR1; KRT18; GATA3; cIAP2; KRT5; RAB27B; IGF1R; HNF3A; CA9; MCM3; STMY3; NPD009; BAD; BBC3; EGFR; CD9; AKT1; CD3z; KRT14; DKFZp564; Bcl2; BECN1; KLK10; DIABLO; MVP; VEGFB; ErbB3; MDM2; Bclx; CDH1; HLA-DPB1; PR; KRT17; GSTp; IRS1; NFKBp65; IGFBP2; RPS6 KB1; DHPS; TIMP3; ZNF217; KIAA1209; COX2; pS2; BRK; CEGP1; EPHX1; VEGF; TP53BP1; COL1A1; FGFR1; and CTSL2, immobilized on a solid surface.

33. The array of claim 32 comprising polynucleotides hybridizing to a plurality of said genes.

34. An array comprising polynucleotides hybridizing to one or more of the following genes: CCND1; EstR1; KRT18; GATA3; cIAP2; KRT5; RAB27B; IGF1R; HNF3A; CA9; MCM3; STMY3; NPD009; BAD; BBC3; EGFR; CD9; AKT1; CD3z; KRT14; DKFZp564; Bcl2; BECN1; KLK10; DIABLO; MVP; VEGFB; ErbB3; MDM2; Bclx; CDH1; HLA-DPB1; PR; KRT17; GSTp; IRS1; NFKBp65; IGFBP2; RPS6 KB1; DHPS; TIMP3; ZNF217; KIAA1209; COX2; pS2; BRK; CEGP1; EPHX1; VEGF; TP53BP1; COL1A1; FGFR1; and CTSL2, immobilized on a solid surface.

35. The array of claim 34 comprising polynucleotides hybridizing to a plurality of said genes.

36. An array comprising polynucleotides hybridizing to one or more of the following genes: VEGFC; B-Catenin; MMP2; MMP9; CNN; FLJ20354; TGFB3; PDGFRb; PLAUR; KRT19; ID1; RIZ1; RAD54L; RB1; SURV; EIF4EL3; CYP2C8; STK15; ACTG2; NEK2; cMet; TIMP2; C20 orf1; DR5; CD31; BIN1; COL1A2; HIF1A; VIM; CDC20; ID2; MCM2; CCNB1; MYH11; Chk2; G-Catenin; HER2; GSN; Ki-67; TOP2A, immobilized on a solid surface.

37. The array of claim 36 comprising polynucleotides hybridizing to a plurality of said genes.

38. The array of any one of claims 32, 34, or 36 wherein said polynucleotides are cDNAs.

39. The array of any one of claims 32, 34, or 36 wherein said polynucleotides are oligonucleotides.

40. The array of any one of claims 32, 34, or 36 comprising at least 5 of said polynucleotides.

41. The array of any one of claims 32, 34, or 36 comprising at least 10 of said polynucleotides.

42. The array of any one of claims 32, 34, or 36 comprising at least 15 of said polynucleotides.

43. The array of any one of claims 32, 34, or 36 comprising polynucleotides hybridizing to all of said genes.

44. The array of any one of claims 32, 34, or 36 comprising more than one polynucleotide hybridizing to the same gene.

45. The array of any one of claims 32, 34, or 36, wherein at least one of said polynucleotides comprises an intron-based sequence the expression of which is correlates with the expression of a corresponding exon sequence.

46. A method of preparing a personalized genomics profile for a patient comprising the steps of:
(a) subjecting RNA extracted from cancer cells obtained from said patient to gene expression analysis;
(b) determining the expression level of at least one gene selected from the group consisting of VEGFC; B-Catenin; MMP2; MMP9; CNN; FLJ20354; TGFB3; PDGFRb; PLAUR; KRT19; ID1; RIZ1; RAD54L; RB1; SURV; EIF4EL3; CYP2C8; STK15; ACTG2; NEK2; cMet; TIMP2; C20 orf1; DR5; CD31; BIN1; COL1A2; HIF1A; VIM; CDC20; ID2; MCM2; CCNB1; MYH11; Chk2; G-Catenin; HER2; GSN; Ki-67; TOP2A; CCND1; EstR1; KRT18; GATA3; cIAP2; KRT5; RAB27B; IGF1R; HNF3A; CA9; MCM3; STMY3; NPD009; BAD; BBC3; EGFR; CD9; AKT1; CD3z; KRT14; DKFZp564; Bcl2; BECN1; KLK10; DIABLO; MVP; VEGFB; ErbB3; MDM2; Bclx; CDH1; HLA-DPB1; PR; KRT17; GSTp; IRS1; NFKBp65; IGFBP2; RPS6 KB1; DHPS; TIMP3; ZNF217; KIAA1209; COX2; pS2; BRK; CEGP1; EPHX1; VEGF; TP53BP1; COL1A1; FGFR1; and CTSL2; wherein the expression level is normalized against a control gene or genes and optionally is compared to the amount found in a corresponding cancer reference tissue set; and
(c) creating a report summarizing the data obtained by said gene expression analysis.

47. The method of claim 46 wherein said cancer cells are obtained from a solid tumor.

48. The method of claim 47 wherein said solid tumor is selected from the group consisting of breast cancer, ovarian cancer, gastric cancer, colorectal cancer, pancreatic cancer, and lung cancer.

49. The method of claim 48 wherein said cancer cells are obtained from a fixed, paraffin-embedded biopsy sample of said tumor.

50. The method of claim 46 wherein said RNA is fragmented.

51. The method of claim 46 wherein said report includes recommendation for a treatment modality for said patient.

52. The method of claim 51 wherein if increased expression of one or more of MMP9; FLJ20354; RAD54L; SURV; CYP2C8; STK15; NEK2; C20 orf1; CDC20; MCM2; CCNB1; Chk2; Ki-67; TOP2A; CCND1; EstR1; KRT18; GATA3; RAB27B; IGF1R; HNF3A; STMY3; NPD009; BAD; BBC3; CD9; AKT1; Bcl2; BECN1; DIABLO; MVP; VEGFB; ErbB3; MDM2; Bclx; CDH1; PR; IRS1; NFKBp65; IGFBP2; RPS6 KB1; DHPS; TIMP3; ZNF217; pS2; BRK; CEGP1; EPHX1; TP53BP1; COL1A1; and FGFR1, or the corresponding expression product is determined, said report includes a prediction that said subject has an increased likelihood of response to chemotherapy.

53. The method of claim 52 further comprising the step of treating said patient with a chemotherapeutic agent.

54. The method of claim 53 wherein said patient is subjected to adjuvant chemotherapy.

55. The method of claim 53 wherein said patient is subjected to neo-adjuvant chemotherapy.

56. The method of claim 55 wherein the neo-adjuvant chemotherapy includes the administration of a taxane derivative.

57. The method of claim 56 wherein the taxane is docetaxel or paclitaxel.

58. The method of claim 56 wherein said chemotherapy further comprises the administration of an additional anti-cancer agent.

59. The method of claim 58 wherein the additional anti-cancer agent is a member of the anthracycline class of anti-cancer agents.

60. The method of claim 59 wherein said additional anti-cancer agent is doxorubicin.

61. The method of claim 58 wherein the additional anti-cancer agent is a topoisomerase inhibitor.

62. The method of claim 51 wherein if increased expression of one or more of VEGFC; B-Catenin; MMP2; CNN; TGFB3; PDGFRb; PLAUR; KRT19; ID1; RIZ1; RB1; EIF4EL3; ACTG2; cMet; TIMP2; DR5; CD31; BIN1; COL1A2; HIF1A; VIM; ID2; MYH11; G-Catenin; HER2; GSN; cIAP2; KRT5; CA9; MCM3; EGFR; CD3z; KRT14; DKFZp564; KLK10; HLA-DPB1; KRT17; GSTp; KIAA1209; COX2; VEGF; and CTSL2, or the corresponding expression product, is determined, said report includes a prediction that said subject has a decreased likelihood of response to chemotherapy.

63. A PCR primer-probe set listed in Table 3.

64. A PCR amplicon listed in Table 4.
